(19) United States
(12) Patent Application Publication
(10) Pub. No.: US 2025/0256117 A1
(43) Pub. Date: Aug. 14, 2025

(54) DEFIBRILLATOR SYSTEM

(71) Applicant: Medtronic, Inc., Minneapolis, MN (US)

(72) Inventors: Paul J. DeGroot, Minneapolis, MN (US); Xusheng Zhang, Shoreview, MN (US); Vladimir P. Nikolski, San Diego, CA (US)

(21) Appl. No.: 18/854,838

(22) PCT Filed: Mar. 31, 2023
(86) PCT No.: PCT/IB2023/053256

§ 371 Date: Oct. 7, 2024

Related U.S. Application Data
(60) Provisional application No. 63/362,862, filed on Apr. 12, 2022.

(51) Int. Cl. A61N 1/39; A61B 5/00; A61B 5/364; A61N 1/05; A61N 1/365; A61N 1/37; G16H 40/63

(52) U.S. Cl. CPC A61N 1/39622; A61B 5/364; A61B 5/4836; A61B 5/686; A61N 1/0573; A61N 1/365; A61N 1/3702; A61N 1/371; A61N 1/3925; G16H 40/63; A61B 2560/0462

(57) ABSTRACT

A medical system configured to monitor physiological symptoms of a patient to detect a current or imminent arrhythmia in a heart of a patient. The medical system is configured to deliver pacing signals to the conduction system of the heart in response to the current or imminent arrhythmia. In examples, the pacing signals are configured to cause high-rate conduction system pacing of the heart. In examples, if the arrhythmia continues or another arrhythmia is detected, the medical system is configured to cause a defibrillation element to deliver a shock to the heart of the patient. In examples, the medical system is configured to deliver a relatively low energy shock following the conduction system pacing.

### TECHNICAL FIELD

The disclosure relates generally to medical device systems and, more particularly, medical device systems configured to monitor cardiac signals and deliver defibrillation therapy.

### BACKGROUND

Some types of medical devices may be used to monitor one or more physiological parameters of a patient, such as cardiac signals, and/or provide therapy to a patient. Such medical devices may include, or may be part of a system that includes, sensors that detect signals associated with such physiological parameters. Values determined based on such signals may be used to assist in detecting changes in patient conditions, in evaluating the efficacy of a therapy, or in general evaluation of patient health. For example, medical devices that monitor physiological parameters may analyze values associated with such physiological parameters in order to identify or monitor a patient condition.

### SUMMARY

This disclosure describes a medical system configured to monitor physiological symptoms of the patient to detect a current or imminent arrhythmia in a heart of the patient. In response to detecting the current or imminent arrhythmia, operating circuitry is configured to cause a first electrode to deliver the pacing signals to the conduction system of the heart, such as the Bundle of His (“His bundle”), Left Bundle Branch (“LBB”), Purkinje fibers, and/or other portions of the conduction system. In examples, the pacing signals are configured to cause high-rate pacing of the heart. If the arrhythmia continues or another arrhythmia is detected, the operating circuitry is configured to cause a defibrillation element to deliver a shock to the heart. In examples, the medical system may be configured to deliver a relatively low energy shock following the conduction system pacing, reducing discomfort to the patient and/or reducing system power expended during the shock.

In an example, a medical system comprises: a first electrode configured to deliver pacing signals to a conduction system of the heart, wherein the first electrode is configured to capture the conduction system of the heart when the first electrode delivers the pacing signals and the first electrode is positioned within tissues of the heart; an electrode support supporting the first electrode, wherein the electrode support is configured to engage tissue of the heart to position the first electrode within the tissues of the heart; a defibrillation element configured to deliver a defibrillation shock to a heart of a patient, wherein the defibrillation element is configured to position within a ventricle of the heart; a monitor device including operating circuitry, wherein the operating circuitry is configured to: monitor one or physiological signals of the patient, detect a current or imminent arrhythmia using the one or more physiological signals, cause, in response to detecting the current or imminent arrhythmia, the first electrode to deliver the pacing signals to the conduction system of the heart, and cause, following delivery of the pacing signals, and if the arrhythmia continues or another arrhythmia is detected, the defibrillation element to deliver a shock to the heart of the patient.

In an example, a method comprises: monitoring, using operating circuitry, one or physiological signals of a patient to detect a current or imminent arrhythmia using the one or more physiological signals; causing, using the operating circuitry, and in response to detecting the current or imminent arrhythmia, a first electrode to deliver pacing signals to a conduction system of the heart, wherein the first electrode is configured to capture the conduction system of the heart when the first electrode delivers the pacing signals and the first electrode is positioned within tissues of the heart, and wherein an electrode support supporting the first electrode is configured to engage tissue of the heart to position the first electrode within the tissues of the heart; and causing, using the operating circuitry, and following delivery of the pacing signals, and if the arrhythmia continues or another arrhythmia is detected by the operating circuitry, a defibrillation element to deliver a shock to the heart of the patient, wherein the defibrillation element is configured to position within a ventricle of the heart.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a conceptual drawing illustrating an example of a defibrillator system.

FIG. 2 shows an example timing diagram for operation of the defibrillator system of FIG. 1.

FIG. 3 is a conceptual drawing illustrating an example of the defibrillator system of FIG. 1 having a first lead and a second lead.

FIG. 4 is a conceptual drawing illustrating an example of the defibrillator system of FIG. 1 having a unitary lead.

FIG. 5 is a conceptual drawing illustrating an example of the defibrillator system of FIG. 1 having a housing configured to be positioned within a heart.

FIG. 6 is a functional block diagram illustrating an example configuration of components of the defibrillator system of FIG. 1.

FIG. 7 is a functional block diagram illustrating an example configuration of the defibrillator system of FIG. 1 including a network.

FIG. 8 is a flowchart illustrating an example process that may be performed by the defibrillator system of FIG. 1.

Like reference characters denote like elements throughout the description and figures.

### DETAILED DESCRIPTION

Cardiac arrest due to ventricular tachyarrhythmia (including ventricular fibrillation (VF) and ventricular tachycardia (VT)) a major cause of death in the United States and worldwide. During VT/VF, disordered electrical activity causes the heart's lower chambers (ventricles) to quiver, or fibrillate, rather than contracting normally. This can prohibit the heart from pumping blood, potentially causing collapse and cardiac arrest. Even with wide deployment of automatic external defibrillators (AEDs), the annual survival rate of people who suffer sudden cardiac arrest is low (e.g., about 5-8% in the United States). For these reasons and others, wide deployment of AEDs alone may still not significantly improve the cardiac arrest survival rate.

Implantable cardiac defibrillators (ICD) may be useful in preventing sudden death in patients at risk for VT/VF. The ICD is typically a battery-powered device placed under the skin of a patient which tracks the heart rate and rhythms. Leads may extend from the ICD into a chamber of the heart (e.g., an atrium and/or ventricle) and/or another location adjacent the heart. (e.g., above or below the ribcage and/or sternum). In some cases, when an abnormal heart rhythm is detected, the CD may deliver cardiac pacing to suppress or convert arrhythmias to sinus rhythm. For example, the ICD may deliver anti-tachycardia pacing (ATP) in an attempt to terminate the arrhythmia. Generally, the ICD is configured to deliver ventricular pacing (e.g., biventricular pacing, left ventricular pacing) reliant on ventricular myocyte propagation of an electrical activation wave front for ventricular activation.

In some cases, when an abnormal heart rhythm is detected, the ICD may deliver an electric shock to restore a normal heartbeat. The ICD may be configured to deliver a relatively high-voltage electrical shock, causing considerable discomfort to the patient, as well as driving component design toward relatively large volume for mechanical packages.

This disclosure describes a medical system configured to detect a current (e.g., currently on-going) or imminent arrhythmia in a heart of a patient. The medical system includes a first electrode configured to deliver pacing signals to the conduction system of the heart, such as the Bundle of His (“His bundle”), Left Bundle Branch (“LBB”), Purkinje fibers, and/or other portions of the conduction system. The medical system includes a defibrillation element (e.g., one or more shock coils, a device housing, and/or other components) configured to deliver a defibrillation shock to the patient. Operating circuitry is configured to monitor physiological status of the patient to detect a current or imminent arrhythmia. The operating circuitry is configured to cause the first electrode to deliver pacing signals to the conduction system to capture the ventricle (e.g., cause ventricular capture). In examples, the pacing signals are configured to cause high-rate pacing of the heart. If the arrhythmia continues or another arrhythmia is detected, the operating circuitry is configured to cause the defibrillation element to deliver a shock to the heart of the patient. In examples, the medical system is configured to deliver a relatively low energy shock following the conduction system pacing, reducing discomfort to the patient.

The pacing signals delivered to the conduction system of the heart (“conduction system pacing”) may more effectively capture the Purkinje network, as opposed to medical systems configured to deliver pacing pulses mainly reliant on ventricular myocyte propagation for ventricular capture. For example, the conduction system pacing may be more effective at influencing and/or impacting the excitable gaps of a re-entrant circuit within the ventricular myocardium. The conduction system pacing may improve the ventricular capture substantially regardless of a specific location of a ventricular tachycardia circuit within the extensive distribution of the Purkinje network or within the myocardium. Hence, the medical system is configured to provide pacing signals (e.g., high rate pacing signals) to the conduction system of the heart when a current or imminent arrhythmia is detected to improve ventricular capture and potentially terminate the ventricular arrhythmia. If the arrhythmia continues or another arrhythmia is detected, the operating circuitry is configured to cause the defibrillation element to deliver a shock to the heart of the patient. The shock may be a lower energy shock than those typically employed, improving comfort to the patient and potentially reducing a general size of any necessary mechanical packaging.

In examples, the medical system is configured to cause the first electrode to deliver high-rate pacing signals to the heart. The high-rate pacing signals may be configured to pace the heart at a heart rate greater than an intrinsic heart rate (e.g., the native heart rate) of the heart. For example, the high-rate pacing signals may be configured to cause a heart rate greater than about 100 beats-per-minute (BPM), another heart rate generally greater than the intrinsic heart rate, or a rate of capture of the conduction system during an ongoing ventricular arrhythmia. In examples, the operating circuitry may be configured to determine the intrinsic heart rate of the heart (e.g., based on a heart rate history of the patient) to deliver the high-rate pacing. In some examples, the operating circuitry may be configured to deliver the high-rate pacing based on pre-programmed instructions, an input provided by a clinician, or another criteria. In some examples, the operating circuitry is configured to ramp the pacing signals toward the high-rate pacing (i.e., decrementing pacing intervals) to assist in ventricular capture using the conduction system pacing. In some examples, the operating circuitry may be configured to deliver a burst therapy (i.e., constant pacing intervals).

The operating circuitry is configured to monitor physiological signals of the patient to determine if the conduction system pacing has impacted (e.g., terminated) the arrhythmia. In examples, the operating circuitry is configured to pause the pacing signals to determine the status of the arrhythmia. If the operating circuitry determines (e.g., based on the physiological signals) that the arrhythmia is continuing and/or a new arrhythmia is detected, the operating circuitry may cause the defibrillation element to deliver a shock to the patient. Hence, the medical system is configured to deliver conduction system pacing to the heart to capture the ventricle when an arrhythmia (e.g., a VT/VF) is detected by the medical system. The medical system is configured to determine the effectiveness of the conduction system pacing, and deliver a shock to the heart based on indications of continuing and/or a new arrhythmia.

By delivering conduction system pacing therapy to the patient in response to an arrhythmia, followed by a shock if necessary, the medical system may reduce an energy level of the shock required as compared to typical defibrillation systems. This can reduce discomfort to the patient as the shock is delivered, and/or reduce the patient stress associated with anticipation of potential shock deliveries. In examples, medical system may be configured to deliver a relatively low energy shock (e.g., a shock less than 5 joules (J)) to the heart following the delivery of the conduction system pacing.

The operating circuitry may be configured to monitor one or more physiological signals such as an electrogram (EGM) and/or electrocardiogram (ECG) of the patient to detect a current or imminent arrhythmia. For example, the operating circuitry may detect, during a first time period, a first pattern in the one or more physiological signals indicative of typical and/or expected cardiac activity of a heart. The operating circuitry may detect, during one or more subsequent time periods, subsequent patterns indicative of a current or imminent arrhythmia. The operating circuitry may cause the first electrode to deliver pacing signals (e.g., high rate pacing signals) to the conduction system of the heart in response to detecting the arrhythmia. The operating circuitry may cause therapy circuitry (e.g., a portion of the operating circuitry) to begin charging in anticipation of the therapy circuitry needing to deliver a shock to the patient following the conduction system pacing.

The operating circuitry may monitor the one or more physiological signals following delivery of the pacing signals to determine if the arrhythmia is continuing and/or a new arrhythmia may be occurring or anticipated to occur. In examples, the operating circuitry is configured to detect a second pattern in the one or more physiological signals during a second time period subsequent to the first period. If the second pattern indicates the arrhythmia has ceased, the operating circuitry may defer and/or decline delivering a shock to the heart using the defibrillation element. If the second pattern indicates the arrhythmia may be continuing, the operating circuitry may cause the defibrillation element to deliver a shock to the heart (e.g., using the shocking circuitry). In some examples, instead of or in addition to causing the defibrillation element to deliver a shock, when the second pattern indicates the arrhythmia may be continuing, the operating circuitry may again cause the first electrode to deliver pacing signals to the conduction system of the heart.

The operating circuitry may substantially continuously monitor the one or more physiological signals of the heart. If at any time, the operating circuitry determines that the arrhythmia has ceased, the operating circuitry may prevent the defibrillation element from delivering a shock to the patient. In some examples, the operating circuitry may additionally notify personnel, such as emergency medical personnel, a clinician, and/or other personnel, that the patient is experiencing or has experienced an arrhythmia.

In examples, the first electrode may be supported (e.g., mechanically supported) by an electrode support configured to position within a chamber (e.g., an atrium or a ventricle) of the heart. The electrode support may be configured to penetrate tissues of the heart to position the first electrode within the tissue (e.g., the myocardial septum) of the heart. The electrode support may be, for example, a helix, a dart, or some other structure configured to penetrate the tissue. The first electrode may be configured to position within the tissues of the heart to deliver pacing pulses to the conduction system of the heart when the electrode support penetrates the tissues of the heart. For example, the electrode support may be configured to penetrate tissues in the ventricular septum of the heart to position the first electrode in proximity to the His bundle, LBB, Purkinje fibers, and/or other portions of the conduction system. The electrode support may be configured to penetrate tissues in the triangle of Koch region of heart to position the first electrode in proximity to the His bundle, LBB, Purkinje fibers, and/or other portions of the conduction system.

In examples, the medical system is configured to displace the defibrillation element from the first electrode when the first electrode penetrates a tissue of the heart (e.g., a myocardial septum or triangle of Koch) and the defibrillation element is positioned within a chamber of the heart. The medical system may displace the defibrillation element from the first electrode such that, following delivery of the conduction system pacing by the first electrode, a subsequent shock from the defibrillation element acts over those portions of the Purkinje network and/or myocardium which might not have been captured by the conduction system pacing. For example, the medical system may be configured to position the defibrillation element in the vicinity of an apex of a ventricle of a heart when the first electrode is positioned to deliver pacing signals to the conduction system of the heart.

The medical system may include a monitor device supporting at least some portion of the operating circuitry. In some examples, the monitor device is configured to position external to the heart when the first electrode and/or defibrillation element are positioned within the heart. For example, the monitor device may be configured for extravascular and/or subcutaneous implantation within the patient (e.g., on the chest of the patient, for example below the clavicle or collarbone). In some examples, the monitor device is configured to reside within a chamber of the heart (e.g., an atrium or a ventricle) when the first electrode and/or defibrillation element are positioned within the heart. In some examples, the operating circuitry is configured to communicate with a patient input/output device (“patient IO device”) and/or a clinician input/output device (“clinician IO device”), such as a mobile phone, a tablet, a workstation, or another IO device. The system may issue a communication indicative of the one or more patient attributes to a clinician input/output device (“clinician IO device”), such as a workstation or other IO device. The operating circuitry may be configured to display a patient status and/or a history of the patient to the patient and/or the clinician. The operating circuitry may include circuitry within the monitor device, the patient IO device, the clinician IO device, and/or other handheld computing devices, computer workstations, servers, and/or other networked computing devices.

The medical system may include one or more return electrodes electrically connected to the operating circuitry. The operating circuitry may be configured to electrically connect the first electrode and the one or more return electrodes. In examples, at least one of the first electrode or the one or more electrodes is configured to electrically connect to the other of the first electrode or the one or more electrodes using at least one of blood of the patient or anatomical structures of the heart when the first electrode is positioned within the heart. In examples, the operating circuitry is configured to electrically connect the defibrillation element and the one or more return electrodes. In examples, at least one of the defibrillation element or the one or more electrodes is configured to electrically connect to the other of the defibrillation element or the one or more electrodes using blood of the patient when the first electrode is positioned within the heart. In some examples, the medical system is configured to position at least one return electrode relative to the defibrillation element to increase and/or establish a displacement between the defibrillation element and at least one return electrode to, for example, more uniformly distribute the energy of a shock vector between the defibrillation element and the at least one return electrode over a ventricular wall of the heart.

As used here, when a first portion of a system supports a second portion of the system, this means that when the second portion causes a first force to be exerted on the first portion, the first portion causes a second force to be exerted on the second portion in response to the first force. The first force and/or second force may be a contact force and/or an action-at-a-distance force. For example, first force and/or second force may be mechanical force, a magnetic force, a gravitational force, or some other type of force. The first portion of the system may be a portion of the system or a portion of a component of the system. The second portion of the system may be another portion of the system or another portion of the same component or a different component. In some examples, when the first portion of the system supports the second portion of the system, this may mean the second portion is mechanically supported by and/or mechanically connected to the first portion.

In some examples, the medical system includes a first lead and a second lead electrically connected to the operating circuitry. A distal portion of the first lead (“first lead distal portion”) may support the electrode support and/or the first electrode. A distal portion of the second lead (“second lead distal portion”) may support the defibrillation element. In examples, the first lead is configured to pass through vasculature of the patient such that at least some portion (e.g., the first lead distal portion) resides within a chamber (e.g., a ventricle) of the heart. The second lead may be configured to pass through vasculature of the patient such that at least some portion (e.g., the second lead distal portion) resides within a chamber (e.g., a ventricle) of the heart. In examples a proximal portion of the first lead (“first lead proximal portion”) and/or a proximal portion of the second lead (“second lead proximal portion”) may be supported by the monitor device when the first lead distal portion and/or second lead distal portion passes through vasculature of the patient.

In examples, the first lead supports a first attachment structure configured to engage heart tissue (e.g., an endocardium or myocardium) within a chamber when the first lead distal portion resides within the chamber. In some examples, the second lead supports a second attachment structure configured to engage heart tissue (e.g., an endocardium or myocardium) within a chamber when the second lead distal portion resides within the chamber. In examples, the electrode support defines the first attachment structure (e.g., when the electrode support defines a helix). The first electrode may be configured to position within tissues of the heart to deliver pacing pulses to the conduction system when the first attachment structure engages tissues within the heart (e.g., tissues of a ventricle or an atrium). The defibrillation element may be configured to position within a ventricle when the second attachment structure engages tissues of the ventricle. The first attachment structure and/or second attachment structure may be, for example, one or more tines, a helical structure, one or more ramped structures, and/or some other structure configured to engage heart tissue.

In some examples, the medical system includes a unitary lead configured to support the first electrode and the defibrillation element. The unitary lead may include a distal portion (“unitary lead distal portion”) opposite a proximal portion (“unitary lead proximal portion”). In examples, the unitary lead is configured to support the first electrode (e.g., via the electrode support) and the defibrillation element using the unitary lead distal portion. The unitary lead may be configured to pass through vasculature of the patient such that at least some portion (e.g., unitary lead distal portion) resides within a chamber of the heart (e.g., a ventricle). The unitary lead proximal portion may be supported by the monitor device when the unitary lead distal portion passes through vasculature of the patient. In examples, the unitary lead includes an attachment structure (“lead attachment structure”) configured to engage heart tissues of the heart to substantially establish and/or maintain the first electrode and/or electrode support within the tissues of the heart. The unitary lead may support the lead attachment structure, the electrode support, and/or the first electrode at a distal end of the medical lead (“unitary distal end”). In examples, the medical lead is configured such that the defibrillation element is positioned within a ventricle when the lead attachment structure is engaged with heart tissues within the ventricle (e.g., tissues of the myocardial septum).

The unitary lead may be configured to position the defibrillation element in proximity to an apex of the ventricle when the lead attachment structure is engaged with heart tissues within the ventricle. In examples, the unitary lead is configured to define a pre-defined shape (e.g., substantially a J shape) when the unitary lead distal portion is positioned in the ventricle and the lead attachment structure is engaged with heart tissues within the ventricle. In some examples, the unitary lead includes a second lead attachment structure configured to position the defibrillation element in proximity to the apex of the ventricle when the lead attachment structure is engaged within the ventricle. The second lead attachment structure may be configured to engage heart tissues to substantially establish, maintain, and/or position the defibrillation element in the vicinity of the apex.

In some examples, the monitor device includes a device housing configured to reside within a chamber of the heart (e.g., a ventricle). The device housing may support at least some portion of the operating circuitry. In examples, the device housing supports a power supply (e.g., battery and/or other power supply) electrically connected to the first electrode and/or the defibrillation element. The power supply may be configured to provide power to the first electrode when the first electrode provides conduction system pacing to the heart. The power supply may be configured to provide power to the defibrillation element when the defibrillation element delivers a shock to the heart. In examples, the monitor device is configured to position within a ventricle of the heart when the first electrode implants within tissues of the ventricle (e.g., a ventricular septum) and the defibrillation element is positioned within the ventricle.

In examples, the monitor device is configured to displace the first electrode from a distal end of the monitor device (“device distal end”), such that the first electrode may implant within tissues of the heart to deliver conduction system pacing. In examples, the monitor device supports the electrode support at the device distal end, and the electrode support provide the displacement. The electrode support may be, for example, a dart structure, a helical structure, and/or some other structure. The electrode support may be configured to penetrate the tissue of the heart to position the first electrode at or in the vicinity of the conduction system of the heart. In examples, the monitor device supports a device lead supporting the defibrillation element. The monitor device may be configured such that monitor device resides within a chamber of the heart when electrode support and defibrillation element reside within in the chamber. In examples, the device lead includes a device lead attachment structure configured to engage tissue in the chamber to cause the defibrillation element to position in proximity to the apex of a ventricle. The device lead attachment structure may be configured to engage heart tissues to substantially establish, maintain, and/or position the defibrillation element in the vicinity of the apex.

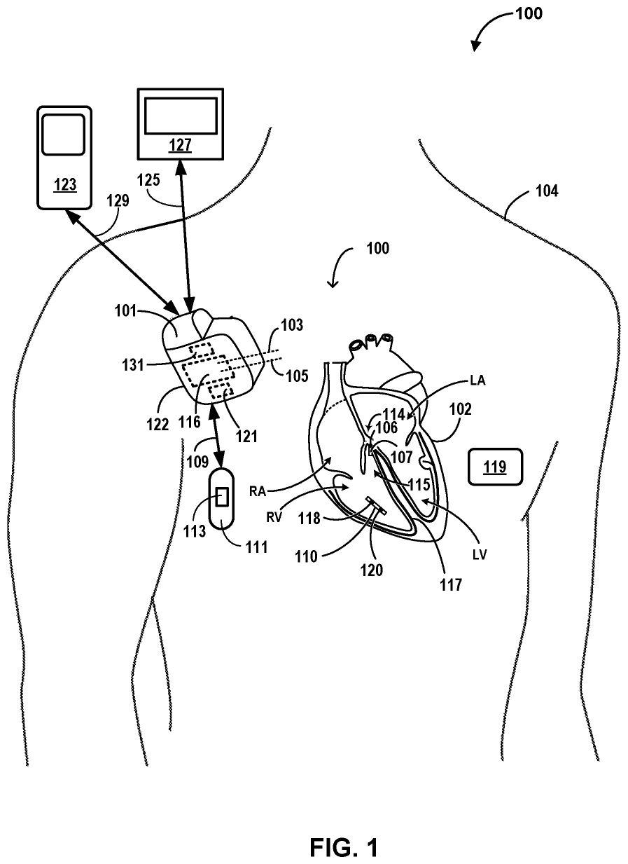

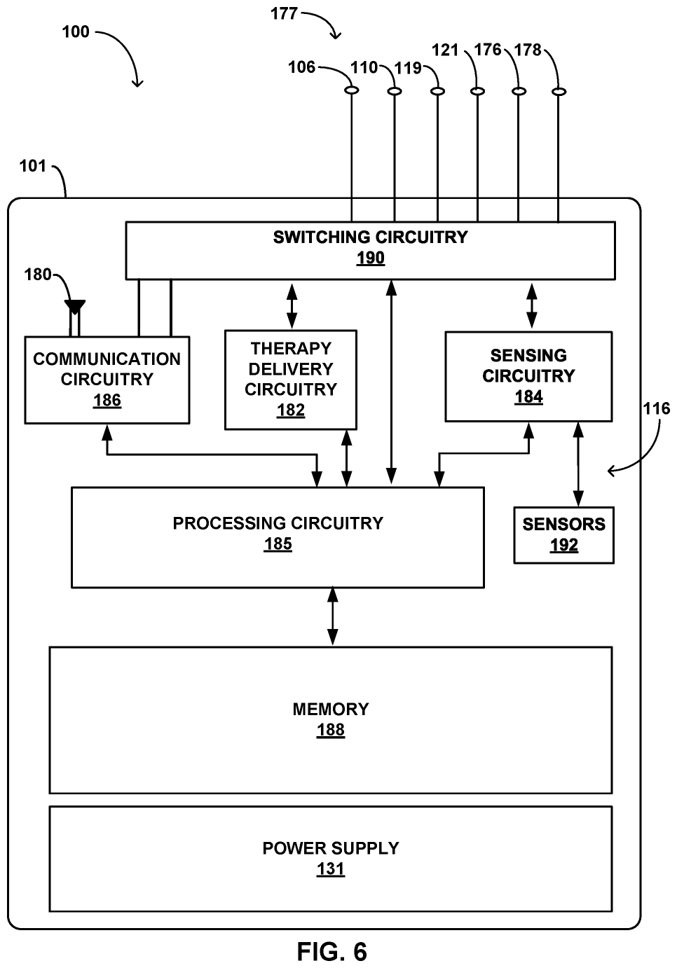

FIG. 1 is a conceptual diagram illustrating a portion of an example medical system 100 configured to deliver conduction system pacing and defibrillation therapy to a heart 102 of a patient 104 using a monitor device 101, a first electrode 106, and a defibrillation element 110. Monitor device 101 may include operating circuitry 116. Operating circuitry 116 may include circuitry illustrated in FIG. 6, such as processing circuitry 185, therapy delivery circuitry 182, and sensing circuitry 184.

Medical system 100 includes a first link 103 configured to electrically connect operating circuitry 116 and first electrode 106. Medical system 100 includes a second link 105 configured to electrically connect operational circuitry 116 and defibrillation element 110. Heart 102 includes a right atrium (RA), a left atrium (LA), a right ventricle (RV), and a left ventricle (LV). Operating circuitry 116 is configured to deliver pacing signals to the conduction system 117 of heart 102 using first electrode 106 (e.g., via first link 103). Operational circuitry 116 is configured to deliver a shock to heart 102 using defibrillation element 110 (e.g., via second link 105).

Medical system 100 may be configured such that first electrode 106 generally implants in heart 102 at a target site such as target site 114 or target site 115 to deliver pacing signals to conduction system 117. First electrode 106 may be configured to deliver the conduction system pacing to, for example, the His bundle, the LBB, Purkinje fibers, and/or other portions of conduction system 117. In examples, target site 114 is a triangle of Koch region in the atrioventricular septal wall of heart 102. Implantation of first electrode 106 in the triangle of Koch region of the atrioventricular septal wall may facilitate pacing of the His bundle or ventricular myocardium. In some examples, target site 115 is in the ventricular septal wall in the basal (e.g., high basal or high septal) region or apical (e.g., low septal or near the apex) region of heart 102. Implantation of first electrode 106 in the basal region of the ventricular septal wall may facilitate pacing of one or both of the His bundle branches. Implantation of first electrode 106 in the apical region may facilitate pacing of Purkinje fibers. Medical system 100 may be configured such that defibrillation element 110 generally positions within a ventricle (e.g., the RV) of heart 102 when first electrode 106 positions within tissues of heart 102 (e.g., at target site 114).

Operating circuitry 116 (e.g., processing circuitry 185) is configured to monitor one or more physiological signals such as an ECM and/or ECG of heart 102 to detect a current or imminent arrhythmia (e.g., a ventricular fibrillation or ventricular tachycardia (VT/VF)). Operating circuitry 116 may cause first electrode 106 to deliver pacing signals to conduction system 117 in response to detecting the current or imminent arrhythmia. In examples, operating circuitry 116 is configured to cause first electrode 106 to deliver high-rate pacing signals to conduction system 117 to capture a ventricle of heart 102 to terminate the arrhythmia. Operating circuitry 116 monitors the one or more physiological signals following delivery of the pacing signals to determine if the arrhythmia is continuing and/or if another arrythmia is anticipated to occur. If the one or more physiological signals indicates the arrhythmia may be continuing, operating circuitry 116 may cause defibrillation element 110 to deliver a shock to heart 102. In examples, the shock is a low energy shock. For example, operating circuitry 116 may cause defibrillation element 110 to deliver a shock of less than about 10 J in some examples, less than about 5 J in some examples, and/or less than about 4 J in some examples. Operating circuitry 116 may be configured to substantially continuously monitor the one or more physiological signals of the heart to detect a current or imminent arrhythmia and/or the cessation of an arrhythmia and/or re-entrant arrhythmia.

Monitor device 101 includes a housing 122 (“device housing 122”). Device housing 122 may support at least some portion of operating circuitry 116. In some examples, monitor device 101 is configured to position external to heart 102 when first electrode 106 and/or defibrillation element 110 are positioned within heart 102. In examples, monitor device 101 is be configured for extravascular and/or subcutaneous implantation within the patient 104. Monitor device 101 may be a wearable device configured to be worn by patient 104. In some examples, monitor device 101 is configured to reside within a chamber of the heart (e.g., an atrium or a ventricle) when first electrode 106 and/or defibrillation element 110 are positioned within the heart. Operating circuitry 116 may include circuitry within monitor device 101, patient IO device 123, clinician IO device 127, and/or other handheld computing devices, computer workstations, servers, and/or other networked computing devices.

First electrode 106 may be configured to deliver pacing signals to achieve physiological ventricular activation through direct capture of the His bundle or bundle branches of conduction system 117. This may allow the pacing signals of first electrode 106 to more effectively capture the Purkinje network, as opposed to electrodes configured to deliver pacing pulses mainly reliant on ventricular myocyte propagation for ventricular capture. In examples, first electrode 106 is configured to penetrate tissue to position within or in close proximity to the His bundle and/or a bundle branch (e.g., the LBB). In examples, medical system 100 is configured to allow a clinician to adjust an implanted position of first electrode 106 in order to position first electrode 106 in a location to deliver the pacing signals to conduction system 117.

For example, medical system 100 may be configured to allow a clinician to alter the position of first electrode 106 based on the one or more physiological signals of patient 104, such as an EGM and/or ECG. Medical system 100 may be configured such that checking (e.g., by a clinician) of the one or more physiological signals at a given implanted position of first electrode 106 may be utilized to evaluate the ventricular capture of first electrode 106 at the given implanted position. Medical system 100 may be configured to substantially secure first electrode 106 (e.g., using electrode support 107 and/or an attachment structure) in an implanted position causing ventricular capture. In some examples, first electrode 106 is configured to selectively capture conduction system 117 with minimum or substantially without capture of surrounding local myocardium.

First electrode 106 may be electrically connected to therapy delivery circuitry of operating circuitry 116. Operating circuitry 116 may be configured to provide electrical signals (e.g., through a conductor) to first electrode 106 (e.g., using the therapy delivery circuitry). First electrode 106 may conduct the electrical signals to conduction system 117, causing the cardiac muscle, e.g., of the ventricles, to depolarize and, in turn, contract. First electrode 106 may also be connected to sensing circuitry of operating circuitry 116, such that operating circuitry may sense activity of heart 102 via first electrode 106. First electrode 106 may have various shapes such as tines, helices, screws, rings, and so on.

First electrode 106 is supported by electrode support 107. Electrode support 107 may be configured to penetrate and/or implant within cardiac tissue at or near a target site such as target site 114, 115 to position first electrode 106 in proximity to conduction system 117. For example, electrode support 107 may be configured to penetrate cardiac tissue to position first electrode 106 at a position at or near the LBB, His bundle, right bundle branch (RBB), other specialized conductive tissue, or other ventricular tissue of heart 102. In examples where first electrode 106 is configured to implant within or in proximity to the LBB or HB, electrode support 107 and/or first electrode 106 may traverse the ventricular septum from right to left. Electrode support 107 may have various shapes such as helices, screws, tines, rings, and so on. Electrode support 107 may define any form sufficient to penetrate and/or implant within tissues of heart 102, such as helices, screws, tines, rings, and so on. In examples, electrode support 107 includes an elongate body defining a helix. In some examples electrode support 107 is an elongate body extending defining a dart (e.g., a substantially straight elongate body). In examples, electrode support 107 defines and/or supports some portion of first link 103 electrically connecting first electrode 106 and operating circuitry 116. For example, electrode support 107 may include an insulated conductor electrically connected to first electrode 106. In examples, an uninsulated portion of electrode support 107 defines at least a portion of first electrode 106.

In examples, operating circuitry 116 is configured to sense one or more physiological signals (e.g., electrical signals) of patient 104 to detect a current or imminent arrhythmia. In examples, operating circuitry 116 is configured to sense the one or more physiological signals by sensing one or more sensing vectors. The sensing vectors sensed by operating circuitry 116 may include and/or be indicative of electrical signals generated by cardiac muscle of heart 102 (e.g., an ECG/EGM). In examples, the sensing vectors are indicative of depolarizations and repolarizations of heart 102 during a cardiac cycle. Operating circuitry 116 may analyze the sensing vectors to detect a current or imminent arrhythmia, such as a VT/VF. For example, operating circuitry 116 may be configured to sense electrical signals via one or more sensing vectors that include first electrode 106, a housing return electrode 121 of device 101, a return electrode 119 configured for cutaneous positioning or implantation within patient 104, electrodes of defibrillation element 110, other electrodes within medical system 100, and/or combinations therein.

In some examples, medical system 100 may include an implantable medical device 111 (“IMD 111”) configured to communicate (e.g., via communication link 109) with operating circuitry 116. IMD 111 may be configured to sense the one or more physiological signals of patient 104 and communicate the one or more signals to operating circuitry 116. IMD 111 may be, for example, a device configured to be implanted (e.g., subcutaneously) outside of a thoracic cavity of patient 104. IMD 111 may be positioned near the sternum near or just below the level of heart 102. IMD 111 may include one or more elements 113 (e.g., electrodes, sensors, and/or circuitry) configured to collectively detect signals enabling operating circuitry 116 to determine and/or detect a current or imminent arrhythmia. For example, IMD 111 may be configured to detect and/or monitor a cardiac signal of heart 102 (e.g., an ECG or EGM) and communicate the cardiac signal to operating circuitry 116. In some examples, IMD 111 may be configured to detect a current or imminent arrhythmia and alert operating circuitry 116 to the current or imminent arrhythmia. IMD 111 may be configured to monitor additional physiological signals and/or parameters influenced by the physiological signals, such as heart rate, blood pressure, heart sounds, acoustic signals, body-temperature, and/or others. In some examples, IMD 111 may be an internal cardiac monitoring device (ICMD), such as the Reveal LINQ™ Insertable Cardiac Monitor, available from Medtronic plc, of Dublin, Ireland.

Operating circuitry 116 is configured to deliver pacing signals to conduction system 117 using first electrode 106 and, if an arrhythmia continues or another arrhythmia is detected, cause defibrillation element 110 to deliver a shock to heart 102. Defibrillation element 110 may be configured to transmit the shock to heart 102 to depolarize cardiac muscles and allow heart 102 (e.g., the sinoatrial node) to re-establish a proper rhythm. Defibrillation element 110 may be configured such that the shock causes a brief electrical current to flow through the heart, depolarizing the cardiac muscles. In examples, medical system 100 is configured such that defibrillation element 110 causes a shock vector substantially across a ventricle of heart 102. The shock vector may extend, for example, from defibrillation element 110 to housing return electrode 121, return electrode 119, and/or another electrode within medical system 100. In some examples, the shock vector may extend from one electrode of defibrillation element 110 to another electrode of defibrillation element 110. As used herein, the term shock may refer to a defibrillation shock, cardioversion shock, and/or other shock delivered to convert an arrhythmia to a sinus rhythm of heart 102.

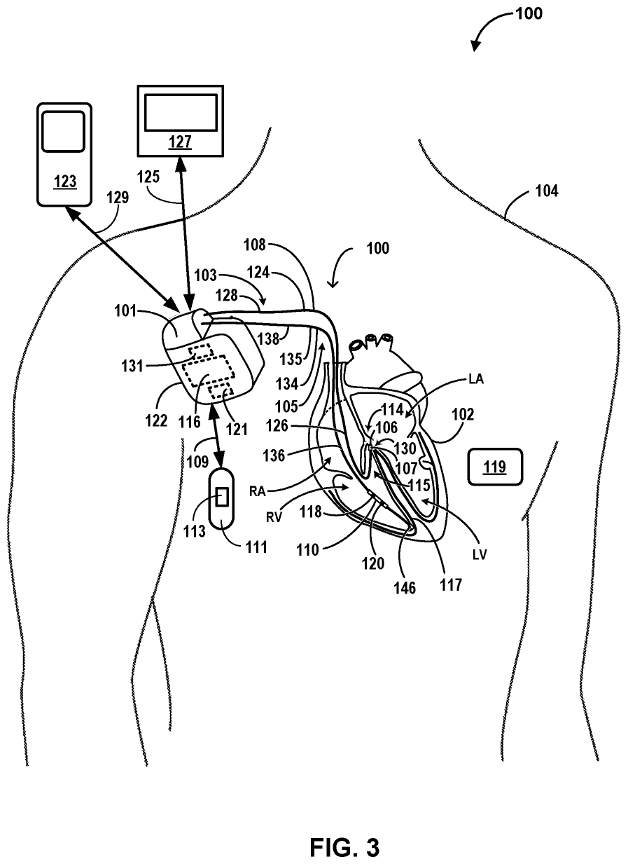

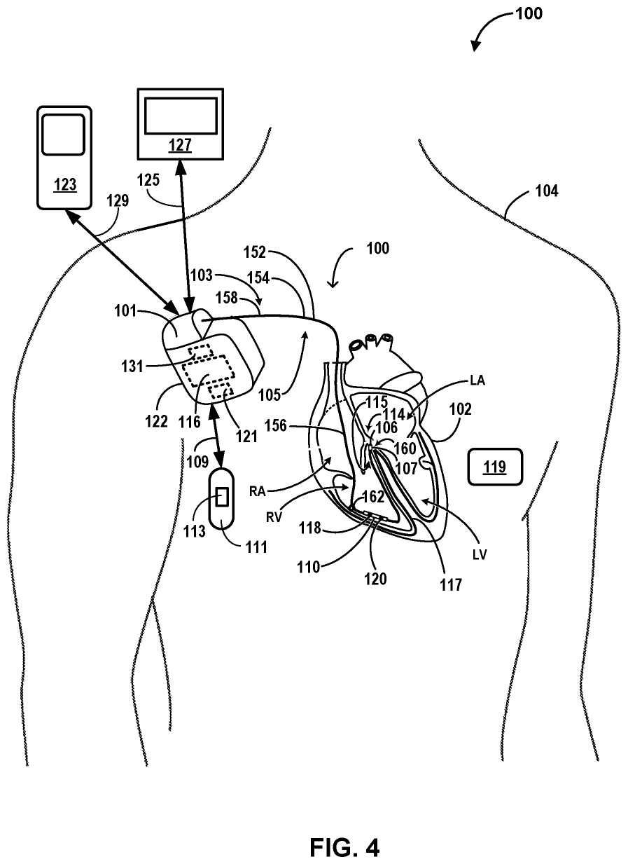

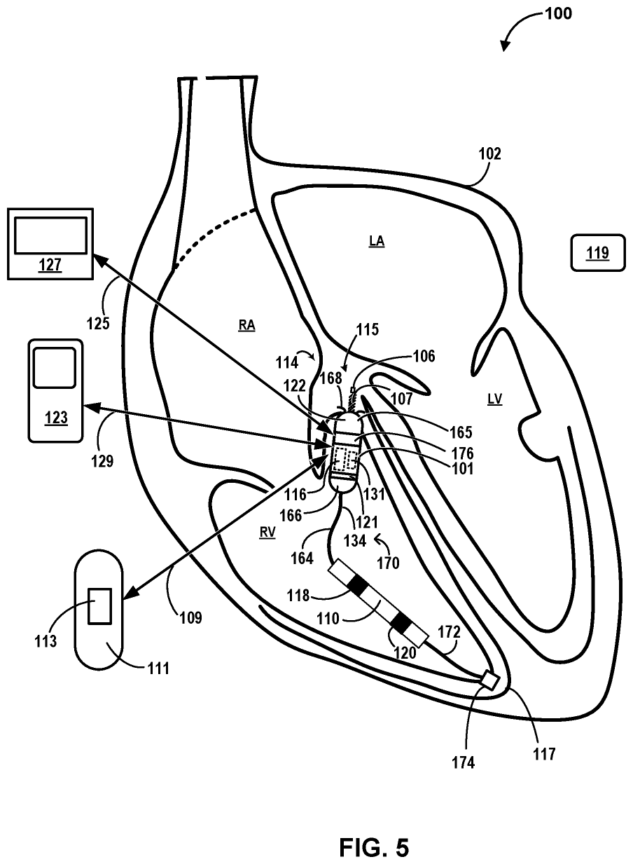

In examples, defibrillation element 110 includes one or more defibrillation electrodes 118 (“defibrillation electrodes 118”). In examples, operating circuitry 116 is configured to activate a defibrillation electrode in defibrillation electrodes 118 concurrently with other defibrillation electrodes in defibrillation electrode 118. In examples, operating circuitry 116 is configured to activate a defibrillation electrode in defibrillation electrodes 118 independently from other defibrillation electrodes in defibrillation electrode 118. Defibrillation electrodes 118 may be electrically connected to operating circuitry 116 via an individual conductor to each individual defibrillation electrode of defibrillation electrodes 118 or via a common conductor electrically connecting two or more defibrillation electrodes of defibrillation electrodes 118. Each individual defibrillation electrode of defibrillation electrodes 118 may be cooperatively and/or independently utilized as part of a shock vector, and/or may be configured for use as an anode or cathode for the shock vector. In some examples, defibrillation electrodes 118 may be configured as one or more coil electrode segments disposed, for example, around an exterior of or within a wall of a lead (e.g., second lead 134 (FIG. 3), unitary lead 152 (FIG. 4), or device lead 164 (FIG. 5). In some examples, one or more of defibrillation electrodes 118 may be a flat ribbon electrode, paddle electrode, braided or woven electrode, mesh electrode, directional electrode, or other type of electrode.

In some examples, defibrillation element 112 may include one or more sensing electrodes 120 (“sensing electrodes 120”). The individual sensing electrodes in sensing electrode 120 may be separated from one another by defibrillation electrodes 118, distal to defibrillation electrodes 118, or proximal to defibrillation electrodes 118. Operating circuitry 116 may be configured to sense the sensing vectors indicative of depolarizations and repolarizations of heart 102 using sensing electrodes 120. Each individual sensing electrode of sensing electrodes 120 may be cooperatively and/or independently utilized as part of a shock vector.

Device housing 122 may support a power supply 131 (e.g., battery and/or other power supply) electrically connected to first electrode 106 and/or defibrillation element 110. Power supply 131 may be configured to provide power to first electrode 106 when first electrode 106 provides conduction system pacing to conduction system 117. Power supply 131 may be configured to provide power to defibrillation element 110 when defibrillation element 110 delivers a shock to heart 102. In examples, monitor device 101 is configured to receive power from an external power supply (not shown) to deliver power to first electrode 106 when first electrode 106 provides conduction system pacing to conduction system 117 and/or to defibrillation element 110 when defibrillation element 110 delivers a shock to heart 102. Power supply 131 and/or the external power supply may be rechargeable and/or non-rechargeable battery. In some examples, device 101 may be configured to be directly coupled to an alternating current or direct current outlet to provide power to first electrode 106 and/or defibrillation electrode 110.

In examples, operating circuitry 116 is configured to communicate (e.g., via communication link 129) with circuitry of a patient IO device 123. Operating circuitry 116 may be configured to communicate (e.g., via communication link 125) with circuitry of a clinician IO device 127. Patient IO device 123 and/or clinician IO device 127 may be any device configured to receive an input from a user and/or provide an output to a user, such as a mobile phone, a tablet, one or more handheld computing devices, computer workstations, servers, other networked computing devices, or another IO device. Medical system 100 may be configured to display the one or more patient attributes or a history of the one or more patient attributes to the patient and/or the clinician using patient IO device 123 and/or clinician IO device 127.

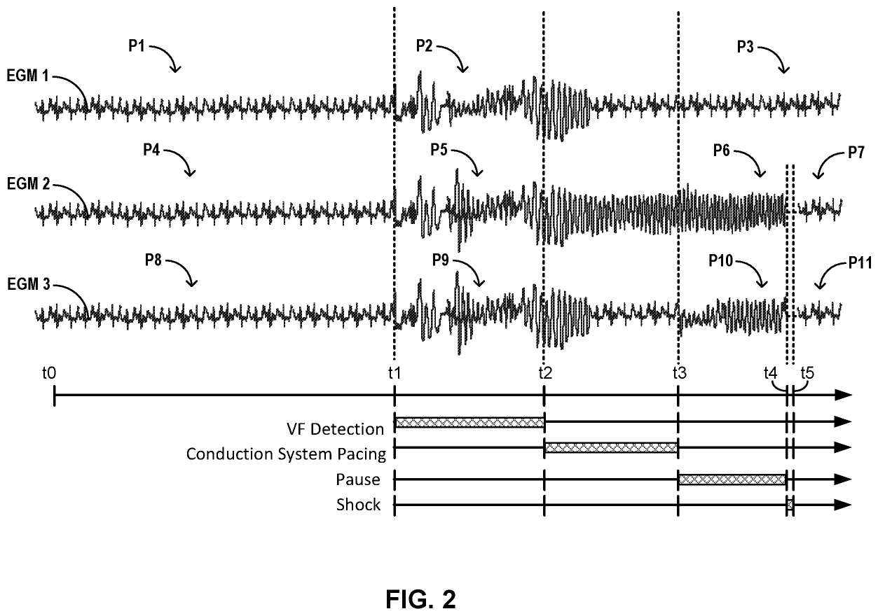

FIG. 2 shows an example timing diagram which might be executed by operating circuitry 116 for the detection a current or imminent arrhythmia (e.g., a VT/VF). Operating circuitry 116 is configured to monitor one or more physiological signals of patient 104 to detect a current or imminent arrhythmia. The one or more physiological signals may include, for example, an ECG or EGM of patient 104. The specific timing and durations used in FIG. 2 represent merely one example, and other timing and durations may also be used. Further, although the example timing diagram is discussed with reference to patterns of an EGM, operating circuitry 116 may be configured to detect a current or imminent arrhythmia using other methods.

FIG. 2 illustrates a first EGM (“EGM 1”), a second EGM (“EGM 2”), and a third EGM (“EGM 3”) which might result from the one or more physiological signals of patient 104. Although presented on similar time scales for illustration, each of EGM 1, EGM 2, and EGM 3 are representative of separate ECM's of heart 102 that may occur over separate chronological time frames. For example, EGM 1 may be an EGM portion occurring over a first time frame, EGM 2 may be an EGM portion occurring over a second time frame different from the first time frame, and EGM 3 may be an EGM portion occurring over a third time frame different from the first time frame and the second time frame. Hence, a time t0, time t1, time t2, time t3, time t4, and time t5 referred to with respect to a first EGM (e.g., EGM 1) is a different time than a time t0, time t1, time t2, time t3, time t4, and time t5 referred to with respect to a second EGM (e.g., EGM 2 and/or EGM 3). Further, although EGM 1, EGM 2, and/or EGM 3 may represent similar EGM morphologies over various time periods for the sake of illustration, EGM 1, EGM 2, and/or EGM 3 may exhibit morphologies different from one another during the various time frames.

Operating circuitry 116 may be configured to detect EGM 1, EGM 2, and/or EGM 3 using first electrode 106, one or more electrodes of defibrillation element 110, housing return electrode 121, return electrode 119, elements 113 of IMD 111, or other sensing electrodes and/or components within medical system 100. In examples, operating circuitry 116 is configured to detect EGM 1, EGM 2, and/or EGM 3 using one or more sensing vectors.

Operating circuitry may be configured to detect an imminent or current arrhythmia (e.g., a VT/VF) based on a departure from a cardiac signal (e.g., an EGM and/or ECG) representative of typical and/or expected cardiac functioning of heart 102. For example, with respect to EGM 1, operating circuitry 116 may detect a first pattern P1 indicative of typical and/or expected cardiac functioning of heart 102. Operating circuitry 116 may detect first pattern P1 during some interval between a time t0 and a time t1. Operating circuitry 116 may monitor EGM 1 on an ongoing basis to confirm that EGM 1 continues to reflect the typical and/or expected cardiac functioning of heart 102.

Operating circuitry 116 may be configured to detect a departure from first pattern P1 to detect and/or predict an imminent or current arrhythmia. For example, operating circuitry 116 may detect a departure of pattern P1 by detecting a pattern P2 in EGM 1 (e.g., at around time t1). Operating circuitry 116 may be configured to detect an imminent arrhythmia based on the departure of pattern P2 from pattern P1. In examples, operating circuitry 116 continues to monitor ECM 1 over a period of from about time t1 to about a time t2 to conduct the arrhythmia detection. Based on the departure of pattern P2 from pattern P1, operating circuitry 116 may determine at time t2 an arrhythmia has been detected.

In response to detecting the arrhythmia, (e.g., at time t2), operating circuitry 116 may be configured to cause first electrode 106 deliver pacing signals to conduction system 117 to, for example, attempt physiological ventricular activation through direct capture of the His bundle or bundle branches of conduction system 117. Operating circuitry 116 may cause first electrode 106 to deliver the conduction system pacing over some time interval, such as from about time t2 to about a time t3. Operating circuitry 116 may be configured to cause first electrode 106 to deliver the pacing signals to conduction system 117 from about time t2 to about time t3. In examples, operating circuitry 116 is configured to cause first electrode 106 to emit a plurality of electrical pulses to cause first electrode 106 to deliver the conduction system pacing signals to conduction system 117. Operating circuitry 116 may be configured to control at least one of an amplitude, a frequency, or a chronological duration of the plurality of electrical pulses to cause first electrode 106 to pace and/or attempt to pace heart 102 to achieve one or more particular cardiac cycles (e.g., heart rates).

In examples, operating circuitry 116 is configured to cause first electrode 106 to deliver high-rate pacing signals to conduction system 117. The high-rate pacing signals may be configured to pace heart 102 at a heart rate greater than an intrinsic heart rate of patient 104. In some examples, operating circuitry 116 is configured to determine the intrinsic heart rate using a portion of EGM 1 (e.g., first pattern P1). In some examples, operating circuitry 116 may be configured to deliver the high-rate pacing signals based on pre-programmed instructions, an input provided by a clinician (e.g., via clinician IO device 127), or another criteria. The high-rate pacing may be configured to cause a heart rate greater than about 100 BPM, another heart rate generally greater than the intrinsic heart rate, or a rate of capture of the conduction system during an ongoing ventricular arrhythmia. For example, during VT/VF, reentrant wavefronts may capture and/or enter the conduction system, causing the rate within the conduction system to be between the intrinsic heart rate and rate at which the VT/VF is driving the conduction system. In examples, the rate at which the VT/VF is driving the conduction system is the rate of capture of the conduction system during the ongoing arrhythmia. In examples, the high-rate pacing may be configured to cause a heart rate greater than the cate of capture of the conduction system during the ongoing arrhythmia.

In some examples, operating circuitry 116 is configured to ramp the pacing signals toward the high-rate pacing to assist first electrode 106 in the ventricular capture. For example, operating circuitry 116 may be configured to control at least one of the amplitude, the frequency, or the chronological duration of the plurality of electrical pulses emitted by first electrode 106 to ramp the pacing signals. In some examples, operating circuitry 116 is configured to cause first electrode 106 to emit electric pulses configured to cause pacing of heart 102 at a first rate. Operating circuitry 116 may be further configured to cause first electrode 106 to emit electric pulses configured to cause pacing of heart 102 at a second rate greater than the first rate. In examples, operating circuitry 116 is configured to ramp (e.g., over a chronological period) the electric pulses emitted by first electrode 106 from the first rate to the second rate. Ramping the electric pulses from the first rate (e.g., the lower rate) toward the second rate (e.g., the higher rate) may assist electrode 106 in ventricular capture using the conduction system pacing.

Operating circuitry 116 is configured to pause the conduction system pacing (e.g., at time t3) to evaluate if the conduction system pacing to conduction system 117 has restored heart 102 to a normal sinus rhythm. Operating circuitry 116 is configured to cause defibrillation element 112 to deliver a shock to heart 102 if the arrhythmia continues or another arrhythmia is detected. Hence, operating circuitry 116 may deliver a shock using defibrillation element 112 following the conduction system pacing, or decline to deliver the shock, based on an assessment of whether the arrhythmia is continuing or another arrhythmia is detected. In examples, rather than pausing the conduction system pacing at the time t3 indicated in FIG. 2, operating circuitry 116 may be configured to pause the conduction system pacing when operating circuitry detects a rhythm of heart 102 similar to and/or indicative of a normal sinus rhythm. For example, operating system 116 may be configured to pause the conduction system pacing prior to the time t3 of FIG. 2 based on detecting a rhythm of heart 102 similar to and/or indicative of a normal sinus rhythm.

For example, operating circuitry 116 may detect a third pattern P3 in EGM 1 subsequent to the conduction system pacing (e.g., subsequent to time t3). Operating circuitry 116 may assess that third pattern P3 is indicative of typical and/or expected cardiac functioning of heart 102. In response to assesses that third pattern P3 is indicative of typical and/or expected cardiac functioning, operating circuitry 116 may decline to deliver a shock to heart 102.

EGM 2 represents a one or more physiological signals of patient 104 wherein operating circuitry 116 might deliver a shock to heart 102 in response to determining the arrhythmia has continued. Operating circuitry 116 may detect a fourth pattern P4 (e.g., from about time t0 to about time t1) indicative of typical and/or expected cardiac functioning, and detect an arrhythmia based on departure of a fifth pattern P5 from fourth pattern P4 (e.g., over the period from time t1 to time t2) in similar manner to that described for EGM 1. Operating circuitry 116 may cause first electrode 106 to deliver pacing signals to conduction system 117 (e.g., over the period from time t2 to time t3) and pause the conduction system pacing (e.g., at time t3) to evaluate if the conduction system pacing has restored heart 102 to a normal sinus rhythm. If operating circuitry 116 detects that the arrhythmia may be continuing (e.g., that the conduction system pacing has not adequately captured the ventricle), operating circuitry 116 may cause defibrillation electrode 110 to deliver a shock to heart 102.

For example, operating circuitry 116 may pause the conduction system pacing at time t3 and detect a sixth pattern P6. Operating circuitry 116 may determine that the arrhythmia of heart 102 is continuing based on sixth pattern P6 (e.g., based on a comparison of sixth pattern P6 and fourth pattern P4). In response to determining that the arrhythmia is continuing, operating circuitry 116 may be configured to deliver a shock to heart 102 between about time t4 and a time t5. Operating circuitry 116 may detect a seventh pattern P7 subsequent to the shock (e.g., subsequent to time t5). Operating circuitry 116 may monitor seventh pattern P7 and decline further action (e.g., additional conduction system pacing and/or shock) if seventh pattern P7 is indicative of typical and/or expected cardiac functioning of heart 102. In examples, operating circuitry 116 may monitor seventh pattern P7 and provide the further action (e.g., additional conduction system pacing and/or shock) if seventh pattern P7 is indicative of continued arrhythmia of heart 102.

EGM 3 represents a one or more physiological signals of patient 104 wherein operating circuitry 116 might deliver a shock to heart 102 in response to determining another arrhythmia has occurred. Operating circuitry 116 may detect an eighth pattern P8 indicative of typical and/or expected cardiac functioning, detect an arrhythmia based on departure of a ninth pattern P9 from eighth pattern P8, cause first electrode 106 to deliver pacing signals to conduction system 117 in response to the departure, and pause the conduction system pacing to evaluate if the conduction system pacing has restored heart 102 to a normal sinus rhythm. If operating circuitry 116 detects another arrhythmia during the pause, operating circuitry 116 may cause defibrillation electrode 110 to deliver a shock to heart 102.

For example, operating circuitry 116 may pause the conduction system pacing at time t3 and detect a tenth pattern P10. Operating circuitry 116 may determine that a re-entry arrhythmia is occurring based on tenth pattern P10 (e.g., based on a comparison of tenth pattern P10 and eighth pattern P8). In response to determining that another arrhythmia may be occurring, operating circuitry 116 may be configured to deliver a shock to heart 102 between about time t4 and time t5. Operating circuitry 116 may detect and monitor an eleventh pattern P11 subsequent to the shock (e.g., subsequent to time t5), and decline further action or provide further action based on eleventh pattern P11.

In examples, operating circuitry 116 is configured to communicate (e.g., via communication link 129) with circuitry of a patient IO device 123. Operating circuitry 116 may be configured to communicate (e.g., via communication link 125) with circuitry of a clinician IO device 127. Patient IO device 123 and/or clinician IO device 127 may be any device configured to receive an input from a user and/or provide an output to a user, such as a mobile phone, a tablet, one or more handheld computing devices, computer workstations, servers, other networked computing devices, or another IO device. Medical system 100 may be configured to display the one or more patient attributes or a history of the one or more patient attributes to the patient and/or the clinician using patient IO device 123 and/or clinician IO device 127.

Operating circuitry 116 may be configured to provide a communication to patient IO device 123 and/or clinician IO device 127 in response to detecting a current or imminent arrhythmia, delivering a shock to heart 102, and/or taking other actions. Operating circuitry 116 may cause patient IO device 123 and/or clinician IO device 127 to generate an output in response to receiving the communication. The output may, for example, be any sort of audio, visual, or haptic signal to alert a user of patient IO device 123 and/or clinician IO device 127 that operating circuitry 116 has detected an arrhythmia, is preparing to deliver a shock, and/or has delivered a shock. In some examples, operating circuitry 116 is configured to solicit input from a user (e.g., using patient IO device 123 and/or clinician TO device 127) that, for example, patient 104 is ready to receive conduction system pacing and/or a shock or that the conduction system pacing and/or shock should be delayed. In some examples, operating circuitry 116 may be configured to recommend actions to patient 104, such as cessation of activity or breathing exercises, for the patient to take to reduce the likelihood of needing a shock.

As discussed, time t0, time t1, time t2, time t3, time t4, time t5, and any time periods therebetween, are exemplary only. Operating circuitry 116 may be configured to define any suitable durations between time t0 and time t1, time t1 and time t2, time t3 and time t3, time t3 and time t4, and time t4 and time t5. Further, the specific sequence and actions discussed with reference to FIG. 2 are exemplary only. Operating circuitry 116 may be configured to take additional actions or fewer actions than those discussed to monitor one or physiological signals of the patient, detect a current or imminent arrhythmia using the one or more physiological signals, cause, in response to detecting the current or imminent arrhythmia, the first electrode to deliver the pacing signals to the conduction system of the heart, and cause, following delivery of the pacing signals, and if the arrhythmia continues or another arrhythmia is detected, the defibrillation element to deliver a shock to the heart of the patient, and/or perform other functions.

FIG. 3 is a conceptual diagram illustrating a portion of medical system 100 configured to deliver conduction system pacing and defibrillation therapy to heart 102 using monitor device 101, first electrode 106 supported (via electrode support 107) by a first lead 108, and defibrillation element 110 supported by a second lead 134. First lead 108 may support electrode support 107. First lead 108 includes an elongate body 124 (“first elongate body 124”) defining a distal portion 126 (“first lead distal portion 126”) and a proximal portion 128 (“first lead proximal portion 128”). First lead 108 may support electrode support 107 using first lead distal portion 126 (e.g., substantially at a distal end of first lead distal portion 126). In examples, first lead 108 is an implantable medical lead extending from monitor device 101 (e.g., device housing 122). First lead 108 may be configured to pass through vasculature of patient 104 such that at least some portion (e.g., first lead distal portion 126) resides within a chamber of heart 102. In examples, monitor device 101 (e.g., device housing 122) supports first lead proximal portion 128. In examples, first lead 108 is configured to allow electrode support 107 and/or first electrode 106 to penetrate tissues in proximity to target site 114, 115 when monitor device 101 is positioned outside of heart 102.

As discussed, electrode support 107 may be configured to penetrate cardiac tissue at or near a target site such as target site 114, 115 to position first electrode 106 in proximity to conduction system 117. First electrode 106 may be electrically connected to a first conductor (not shown) extending through and/or within first lead 108 (e.g., through first elongate body 124). In examples, the first conductor defines first link 103. The first conductor may be configured to electrically connect first electrode 106 to therapy delivery circuitry of operating circuitry 116.

In examples, electrode support 107 is configured to extend distally from first lead distal portion 126 (e.g., from a distal end of first lead distal portion). Electrode support 107 may be mechanically connected to first lead distal portion 126, such that a distal translation of first lead distal portion 126 causes a corresponding distal translation of electrode support 107 and a proximal translation of first lead distal portion 126 causes a corresponding proximal translation of electrode support 107. Electrode support 107 may be mechanically connected to first lead distal portion 126 such that a rotation of first lead distal portion 126 about an axis (e.g., a longitudinal axis of first lead distal portion 126) causes a corresponding rotation of electrode support 107. First electrode 106 may be configured to distally translate, proximally translate, and/or rotate when first lead distal portion 126 causes a distal translation, a proximal translation, and/or a rotation of electrode support 107. In some examples, electrode support 107 is configured to translate distally and/or translate proximally relative to first lead distal portion 126 such that, for example, a length from first lead distal portion 126 (e.g., from the distal end of first lead distal portion 126) to first electrode 106 may be adjusted based on an anatomy of patient 104.

In some examples, first lead 108 supports a first attachment structure 130 configured to engage heart tissue within a chamber of heart 102 when first lead distal portion 126 resides within the chamber. First attachment structure 130 may be configured to engage the heart tissue to substantially maintain and/or establish electrode support 107 and/or first electrode 106 within the tissues of heart 102. First attachment structure 130 may be, for example, one or more tines, a helical structure, one or more ramped structures, and/or some other structure configured to engage heart tissue. In some examples, electrode support 107 defines first attachment structure 130 (e.g., when electrode support 107 defines a helix).

Medical system 100 may include a second lead 134 supporting defibrillation element 110. Second lead 134 includes an elongate body 135 (“second elongate body 135”) defining a second lead distal portion 136 and a second lead proximal portion 138. In examples, second lead 134 is an implantable medical lead extending from monitor device 101 (e.g., device housing 122). Second lead 134 may be configured to pass through vasculature of patient 104 such that at least some portion (e.g., second lead distal portion 136) resides within a chamber of heart 102. In examples, monitor device 101 (e.g., device housing 122) supports second lead proximal portion 138. In examples, second lead 134 is configured to position defibrillation element 110 within a chamber (e.g., a ventricle) of heart 102 when monitor device 101 is positioned outside of heart 102.

Defibrillation element 110 may be electrically connected to a second conductor (not shown) extending through and/or within second lead 134 (e.g., through second elongate body 135). In examples, the second conductor defines second link 105. The second conductor may be configured to electrically connect defibrillation element 112 to therapy circuitry of operating circuitry 116.

Second lead 134 may support a second attachment structure 146 configured to engage heart tissue within a chamber of heart 102 when second lead distal portion 136 resides within the chamber. Second attachment structure 146 may be, for example, one or more tines, a helical structure, one or more ramped structures, and/or some other structure configured to engage heart tissue. In examples, second lead 134 supports second attachment structure 146 at a distal end of second lead 134. In examples, second lead 134 is configured to position defibrillation element 110 in the vicinity of an apex of a ventricle (e.g., an apex of the RV) when second attachment structure 146 engages tissues within the apex of the ventricle.

FIG. 4 is a conceptual diagram illustrating a portion of medical system 100 configured to deliver conduction system pacing and defibrillation therapy to heart 102 using monitor device 101 and a unitary lead 152 supporting first electrode 106 (via electrode support 107) and defibrillation element 110. Unitary lead 152 may support electrode support 107. Unitary lead 152 includes an elongate body 154 (“unitary lead elongate body 154”) defining a distal portion 156 (“unitary lead distal portion 156”) and a proximal portion 158 (“unitary lead proximal portion 158”). Unitary lead 152 may support electrode support 107 using unitary lead distal portion 156 (e.g., substantially at a distal end of unitary lead distal portion 156). In example, electrode support 107 is configured to extend distally from unitary lead distal portion 156 (e.g., from the distal end of unitary lead distal portion).

Electrode support 107 may be mechanically connected to unitary lead distal portion 156, such that a distal translation of unitary lead distal portion 156 causes a corresponding distal translation of electrode support 107 and a proximal translation of unitary lead distal portion 156 causes a corresponding proximal translation of electrode support 107. Electrode support 107 may be mechanically connected to unitary lead distal portion 156 such that a rotation of unitary lead distal portion 156 about an axis (e.g., a longitudinal axis of unitary lead 152) causes a corresponding rotation of electrode support 107. First electrode 106 may be configured to distally translate, proximally translate, and/or rotate when unitary lead distal portion 156 causes a distal translation, a proximal translation, and/or a rotation of electrode support 107. In some examples, electrode support 107 is configured to translate distally and/or translate proximally relative to unitary lead distal portion 156 such that, for example, a length from unitary lead distal portion 156 (e.g., from the distal end of unitary lead distal portion 156) to first electrode 106 may be adjusted based on an anatomy of patient 104.

In examples, unitary lead 152 is an implantable medical lead extending from monitor device 101 (e.g., device housing 122). Unitary lead 152 may be configured to pass through vasculature of patient 104 such that at least some portion (e.g., unitary lead distal portion 156) resides within a chamber of heart 102. In examples, monitor device 101 (e.g., device housing 122) supports unitary lead proximal portion 158. In examples, unitary lead 152 is configured to allow electrode support 107 and/or first electrode 106 to penetrate tissues in proximity to target site 114, 115 when monitor device 101 is positioned outside of heart 102. In examples, unitary lead 152 includes the first conductor electrically connecting first electrode 106 to operating circuitry 116. Unitary lead 152 may include the second conductor electrically connecting defibrillation element 110 to operating circuitry 116. In examples, unitary lead 152 (e.g., the first conductor) defines first link 103. Unitary lead 152 (e.g., the second conductor) may define second link 105.

Unitary lead 152 may support an attachment structure 160 (“lead attachment structure 160”) configured to engage heart tissue within a chamber of heart 102 when unitary lead distal portion 156 resides within the chamber. Lead attachment structure 160 may be configured to engage the heart tissue to substantially maintain and/or establish electrode support 107 and/or first electrode 106 within the tissues of heart 102. Lead attachment structure 160 may be, for example, one or more tines, a helical structure, one or more ramped structures, and/or some other structure configured to engage heart tissue. In some examples, electrode support 107 defines lead attachment structure 160 (e.g., when electrode support 107 defines a helix). Unitary lead 152 may be configured such that defibrillation element 110 is positioned within a ventricle of heart 102 when lead attachment structure 160 is engaged with heart tissues within the ventricle.

Unitary lead 152 may be configured to position defibrillation element 110 in proximity to an apex of a ventricle (e.g., a RV) when lead attachment structure 160 is engaged with heart tissues within the ventricle. In examples, unitary lead 152 is configured to define a pre-defined shape (e.g., substantially a J shape) when unitary lead distal portion 156 is positioned in the ventricle and lead attachment structure 160 is engaged with heart tissues within the ventricle. In some examples, the unitary lead 152 (e.g., unitary lead distal portion 156) includes a second lead attachment structure 162 configured to position defibrillation element 110 in proximity to the apex of the ventricle when lead attachment structure 160 is engaged to tissue within the ventricle (e.g., in proximity to target site 114, 115). Second lead attachment structure 162 may be configured to engage heart tissues to substantially establish, maintain, and/or position defibrillation element 110 in the vicinity of the apex. Second lead attachment structure 162 may be, for example, one or more tines, a helical structure, one or more ramped structures, and/or some other structure configured to engage heart tissue.

FIG. 5 is a conceptual diagram illustrating a portion of an example medical system 100 configured to deliver conduction system pacing and defibrillation therapy to heart 102 with monitor device 101 positioned within a chamber (e.g., a ventricle) of heart 102. Device housing 122 may be configured to reside within the chamber of heart 102 when first electrode 106 and/or defibrillation element 110 positions within a chamber or septum of heart 102. Monitor device 101 may be configured such that first electrode 106 may position in proximity to conduction system 117 (e.g., in proximity to target site 114, 115) when device housing 122 is positioned within the chamber of the heart. Monitor device 101 may be configured such that device housing 122 supports a device lead 164 and/or defibrillation element 110 within the ventricle when first electrode 106 positions in proximity to conduction system 117. Hence, in some examples, medical system 100 may be configured such that device housing 122, electrode support 107, defibrillation element 110, and/or device lead 164 may be positioned within a chamber of the heart (e.g., a ventricle) when first electrode 106 is positioned in proximity to conduction system 117. In examples, device housing 122 is configured to position within the RV when electrode support 107, defibrillation electrode 110, and device lead 164 position within the RV.

In examples, device housing 122 defines a distal portion 165 (“housing distal portion 165”) and a proximal portion 166 (“housing proximal portion 166”) opposite housing distal portion 165. Monitor device 101 may be configured such that housing distal portion 165 supports electrode support 107. In examples, electrode support 107 is configured to extend distally from housing distal portion 165. Electrode support 107 may be mechanically connected to housing distal portion 165, such that a distal translation of device housing 122 causes a corresponding distal translation of electrode support 107 and a proximal translation of device housing 122 causes a corresponding proximal translation of electrode support 107. Electrode support 107 may be mechanically connected to housing distal portion 165 such that a rotation of device housing 122 about an axis (e.g., a longitudinal axis of device housing 122) causes a corresponding rotation of electrode support 107.

First electrode 106 may be configured to distally translate, proximally translate, and/or rotate when device housing 122 causes a distal translation, a proximal translation, and/or a rotation of electrode support 107. In some examples, electrode support 107 is configured to translate distally and/or translate proximally relative to device housing 122 such that, for example, a length from housing distal portion 165 to first electrode 106 may be adjusted based on an anatomy of patient 104 (FIG. 1). In examples, electrode support 107 defines and/or includes the first conductor electrically connecting first electrode 106 and operating circuitry 116. In examples, the first conductor and/or electrode support 107 defines and/or includes first link 103 (FIG. 1).

In some examples, device housing 122 (e.g., housing distal portion 165)) supports an attachment structure 168 (“device attachment structure 168”) configured to engage heart tissue within a chamber of heart 102 when device housing 122 resides within the chamber. The device attachment structure 168 may be configured to engage the heart tissue to substantially maintain and/or establish electrode support 107 and/or first electrode 106 within the tissues of heart 102. Device attachment structure 168 may be, for example, one or more tines, a helical structure, one or more ramped structures, and/or some other structure configured to engage heart tissue. In some examples, electrode support 107 defines device attachment structure 168 (e.g., when electrode support 107 defines a helix). Device housing 122 may be configured such that defibrillation element 110 is positioned within a ventricle of heart 102 when device attachment structure 168 is engaged with heart tissues within the ventricle.

In examples, device lead 164 includes a distal portion 170 (“device lead distal portion 170”) and a proximal portion 172 (“device lead proximal portion 172”). Monitor device 101 (e.g., device housing 122) may support device lead distal portion 170. In examples, device lead 164 includes the second conductor electrically connecting defibrillation element 110 to operating circuitry 116. In examples, device lead 164 and/or the second conductor defines second link 105 (FIG. 1).

Device lead 164 (e.g., device lead proximal portion 172) may support an attachment structure 174 (“device lead attachment structure 174”) configured to engage heart tissue within a chamber of heart 102 (e.g., a ventricle) when device lead 164 and/or device housing 122 resides within the chamber. Device lead attachment structure 174 may be for example, one or more tines, a helical structure, one or more ramped structures, and/or some other structure configured to engage heart tissue. In examples, device lead 164 supports device lead attachment structure 174 at a distal end of device lead 164. In examples, device lead 164 is configured to position defibrillation element 110 in the vicinity of an apex of a ventricle (e.g., an apex of the RV) when device lead attachment structure 174 engages tissues within the apex of the ventricle.

In examples, monitor device 101 includes a shock return electrode 176. Medical system 100 is configured such that defibrillation element 110 (e.g., defibrillation electrodes 118) causes a shock vector substantially across a ventricle of heart 102 to shock return electrode 176, and/or to housing return electrode 121, return electrode 119 (FIG. 1), and/or another electrode within medical system 100. Shock return electrode 176 may be electrically connected to operating circuitry 116 via an individual conductor to each individual defibrillation electrode of defibrillation electrodes 118 or via a common conductor electrically connecting two or more defibrillation electrodes of defibrillation electrodes 118. In examples, monitor device 101 is configured to position shock return electrode 176 relative to defibrillation element 110 to increase and/or establish a displacement between defibrillation element 110 and shock return electrode 176 to, for example, more uniformly distribute the energy of a shock vector between defibrillation element 110 and shock return electrode 176 over a ventricular wall of heart 102. In examples, housing distal portion 165 supports shock return electrode 176 and housing proximal portion 166 supports device lead distal portion 170.

FIG. 6 is a functional block diagram illustrating an example configuration of medical system 100 of FIGS. 1 and 3-5 in accordance with one or more examples and/or techniques described herein. Medical system 100 includes electrodes 177 including first electrode 106, defibrillation element 110 (e.g., defibrillation electrode 118 and/or sensing electrodes 120), return electrode 119, housing return electrode 121, shock return electrode 176, and/or other electrodes 178 within medical system 100. Medical system 100 may include antenna 180, therapy delivery circuitry 182, operating circuitry 116, sensing circuitry 184, processing circuitry 185, communication circuitry 186, memory 188, switching circuitry 190, sensors 192, and/or power supply 131. In some examples, memory 188 includes computer-readable instructions that, when executed by operating circuitry 116, cause operating circuitry 116 to perform various functions attributed to operating circuitry 116 herein. Memory 188 may include any volatile, non-volatile, magnetic, optical, or electrical media, such as a random access memory (RAM), ferroelectric RAM (FRAM), read-only memory (ROM), non-volatile RAM (NVRAM), electrically-erasable programmable ROM (EEPROM), flash memory, or any other digital media.

Operating circuitry 116 (e.g., therapy delivery circuitry 182, sensing circuitry 184, processing circuitry 185, and/or switching circuitry 190) may include fixed function circuitry and/or programmable operating circuitry. Operating circuitry 116 may include any one or more of a microprocessor, a controller, a digital signal processor (DSP), an application specific integrated circuit (ASIC), a field-programmable gate array (FPGA), or equivalent discrete or analog logic circuitry. In some examples, operating circuitry 116 may include multiple components, such as any combination of one or more microprocessors, one or more controllers, one or more DSPs, one or more ASICs, or one or more FPGAs, as well as other discrete or integrated logic circuitry. The functions attributed to operating circuitry 116 herein may be embodied as software, firmware, hardware or any combination thereof. Therapy delivery circuitry 182, sensing circuitry 184, processing circuitry 185, and/or switching circuitry 190 may have attributes of and/or perform any functions of operating circuitry 116.

Sensing circuitry 184 and communication circuitry 186 may be selectively coupled to electrodes 177 via switching circuitry 190, as controlled by operating circuitry 116. Sensing circuitry 184 may monitor signals from electrodes 177 in order to monitor electrical activity of heart 102 (e.g., to produce an ECG/EGM), deliver pacing signals to conduction system 117, deliver a shock to heart 102, and/or perform other functions related to one or more physiological signals of patient 104. Sensing circuitry 184 also may monitor signals from sensors 192, which may include impedance sensors, accelerometers, a gyroscope, a PPG sensor, a tissue oxygen sensor, a blood pressure monitor, respiration rate sensors, respiration effort sensors, respiration pattern sensor, a temperature sensor, or other such sensors configured to monitor one or more physiological signals of patient 104. In some examples, sensing circuitry 184 may include one or more filters and amplifiers for filtering and amplifying signals received from one or more of electrodes 177 and/or sensors 192.

Therapy delivery circuitry 182 is configured to deliver, via electrodes 177, therapy to patient 104. In examples, therapy delivery circuitry 182 is configured to deliver conduction system pacing to patient 104 via first electrode 106. Therapy delivery circuitry 182 may be configured to deliver a shock to patient 104 via defibrillation element 110. Therapy delivery circuitry 182 may, for instance, include a variety of capacitors, transformers, switches, and the like configured to deliver a conduction system pacing and/or a shock to the heart 102 via electrodes 177.

Communication circuitry 186 may include any suitable hardware, firmware, software or any combination thereof for communicating with another device, such as patient IO device 123 and/or clinician IO device 127. Communication circuitry 186 may be configured to communicate using communication links 109, 125, 129 (FIGS. 1, 3-5) and/or others. Under the control of operating circuitry 116, communication circuitry 186 may receive downlink telemetry from, as well as send uplink telemetry to, another device (e.g., with the aid of antenna 180). In addition, operating circuitry 116 may communicate with a networked computing device and a computer network, such as the Medtronic CareLink® Network developed by Medtronic, plc, of Dublin, Ireland.

A clinician or other user may retrieve data from operating circuitry 116 using clinician IO device 127, patient IO device 123, and/or another local or networked computing device configured to communicate with operating circuitry 116 via communication circuitry 186. The clinician may also program parameters of operating circuitry 116 using clinician IO device 127, patient IO device 123, and/or another local or networked computing device.

Power supply 131 is configured to deliver operating power to the components of medical system 100. Power supply 131 may include one or more batteries and a power generation circuit to produce the operating power. In some examples, the one or more batteries may include a battery that is rechargeable to allow extended operation. In some examples, recharging is accomplished through proximal inductive interaction between an external charger and an inductive charging coil within medical system 100 (e.g., within device housing 122 (FIGS. 1, 3-5). Power supply 131 may include any one or more of a plurality of different battery types, such as nickel cadmium batteries and lithium ion batteries. A non-rechargeable battery may be selected to last for several years, while a rechargeable battery may be inductively charged from an external device, e.g., on a daily or weekly basis. In some examples, power supply 131 may include both a rechargeable battery and a non-rechargeable battery.

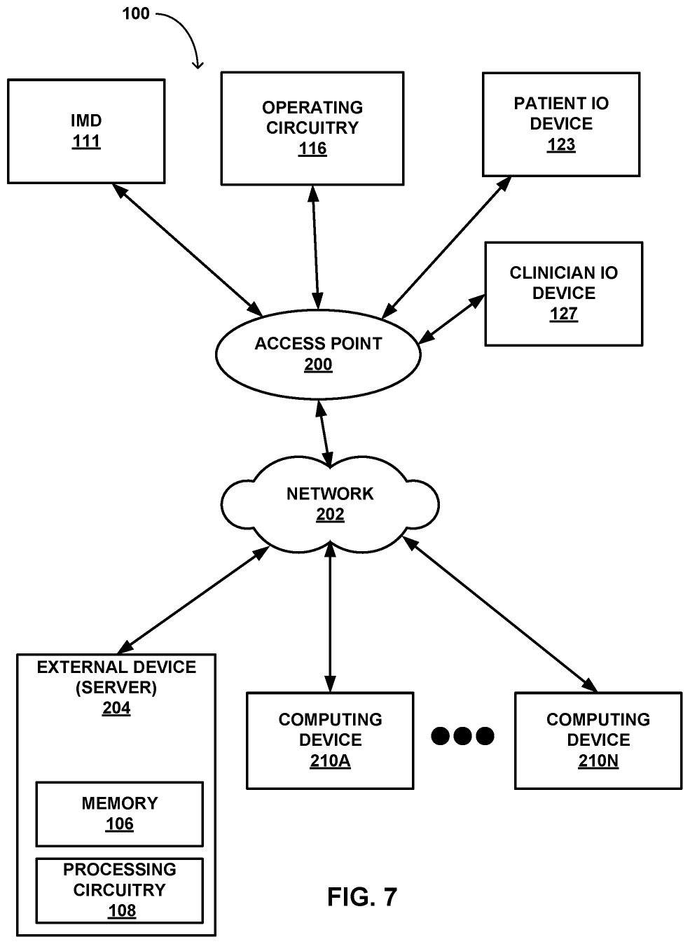

FIG. 7 is a block diagram illustrating an example system that includes operating circuitry 116, patient IO device 123, clinician IO device 127, IMD 111, an access point 200, a network 202, an external device 204 such as a server, and one or more other computing devices 210A-210N. Any of operating circuitry 116, patient IO device 123, clinician IO device 127, IMD 111, external device 204, and/or computing devices 210A-210N may be coupled to any other of operating circuitry 116, patient IO device 123, clinician IO device 127, IMD 111, external device 204, and/or computing devices 210A-210N via network 202 and/or access point 200, in accordance with one or more techniques described herein.

Access point 200 may include a device that connects to network 202 via any of a variety of connections, such as telephone dial-up, digital subscriber line (DSL), or cable modem connections. In other examples, access point 200 may be coupled to network 202 through different forms of connections, including wired or wireless connections. In some examples, access point 200 may be a user device (e.g., patient IO device 123), such as a tablet or smartphone, that may be co-located with patient 104. As discussed above, operating circuitry 116 may be configured to transmit data, such as current values and heart failure statuses, to external device 204 and/or external computing devices 210A-210N. In addition, access point 200 may interrogate operating circuitry 116, such as periodically or in response to a command from patient IO device 123, clinician IO device 127, IMD 111, and/or network 202, in order to retrieve patient data determined by operating circuitry 116. Access point 200 may then communicate the retrieved data to external device 204 (e.g., a server) via network 202.

In some cases, external device 204 may be configured to provide a secure storage site for data that has been collected from operating circuitry 116 and/or other devices within medical system 100. In some cases, external device 204 may assemble data in web pages or other documents for viewing by trained professionals, such as clinicians, via computing devices 210A-210N. In examples, external device 204 may be used to store data indicative of instructions for execution by operating circuitry 116. External device 204 may be used by software or applications running on IMD 111, patient IO device 123, clinician IO device 127, computing devices 210A-210N, to, for example, temporarily store information during program execution.

One or more aspects of the illustrated system of FIG. 7 may be implemented with general network technology and functionality, which may be similar to that provided by the Medtronic CareLink® Network developed by Medtronic plc, of Dublin, Ireland.

In some examples, one or more of computing devices 210A-210N (e.g., device 210A) and/or clinician IO device 127 may be a tablet or other smart device located with a clinician, by which the clinician may program, receive alerts from, and/or interrogate operating circuitry 116. For example, the clinician may access parameter values associated with patient 104 through computing device 210A and/or clinician IO device 127, such as when patient 104 is in in between clinician visits, to check on a status of patient 104 as desired. In some examples, the clinician may enter instructions for a medical intervention for patient 104 into computing device 210A and/or clinician IO device 127. Computing device 210A and/or clinician IO device 127 then may transmit the instructions for medical intervention to another of computing devices 210A-210N (e.g., device 210B) and/or to operating circuitry 116. For example, such instructions for medical intervention may include an instruction to change a drug dosage, timing, or selection, to schedule a visit with the clinician, or to seek medical attention.

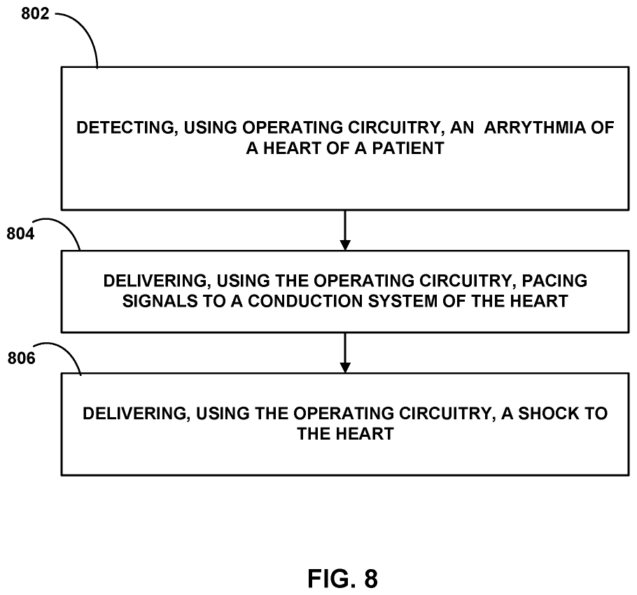

A technique for using medical system 100 in FIG. 8. Although the technique is described mainly with reference to medical system 100 of FIGS. 1 and 3-7, the technique may be applied to other medical systems in other examples.

The technique includes monitoring, using operating circuitry 116, one or physiological signals of patient 104 to detect a current or imminent arrhythmia of heart 102 using the one or more physiological signals (802). Operating circuitry 116 monitor the one or more physiological signals using first electrode 106, one or more electrodes of defibrillation element 110, housing return electrode 121, return electrode 119, elements 113 of IMD 111, or other sensing electrodes and/or components within medical system 100. Operating circuitry 116 may monitor the one or more physiological signals using one or more sensing vectors between any two or more of first electrode 106, one or more electrodes of defibrillation element 110, housing return electrode 121, return electrode 119, elements 113 of IMD 111, or other sensing electrodes and/or components within medical system 100. In examples, operating circuitry 116 detects the imminent or current arrhythmia (e.g., a VT/VF) based on a departure from a cardiac signal (e.g., an EGM and/or ECG) representative of typical and/or expected cardiac functioning of heart 102. For example, operating circuitry 116 may detect a first pattern P1 indicative of typical and/or expected cardiac functioning of heart 102. Operating circuitry 116 detect the imminent or current arrhythmia based on a departure of a second pattern P2 from the first pattern P1.

The technique includes causing, using operating circuitry 116, and in response to detecting the current or imminent arrhythmia, first electrode 106 to deliver pacing signals to conduction system 117 of heart 102 (804). First electrode 106 may capture conduction system 117 of heart 102 when first electrode 106 delivers the pacing signals and first electrode 106 is positioned within tissues of heart 102. In examples, first electrode 106 delivers conduction system pacing to, for example, the His bundle, the LBB, Purkinje fibers, and/or other portions of conduction system 117. First electrode 106 may be positioned and/or implanted within or in proximity to conduction system 117. In examples, first electrode 106 is positioned and/or implanted within or in proximity to target site 114, 115. In examples, an electrode support 107 supporting first electrode 106 causes first electrode 106 to implant within or in proximity to conduction system 117.

Operating circuitry 116 may cause first electrode 106 to deliver high-rate pacing signals to conduction system 117. The high-rate pacing signals may pace heart 102 at a heart rate greater than the intrinsic heart rate of patient 104. In examples, operating circuitry 116 determines the intrinsic heart rate. In examples, operating circuitry 116 delivers the high-rate pacing signals based on pre-programmed instructions, an input provided by a clinician (e.g., via clinician IO device 127), or another criteria. The high-rate pacing may cause a heart rate greater than about 100 BPM, or another heart rate generally greater than the intrinsic heart rate, or a rate of capture of the conduction system during an ongoing ventricular arrhythmia. In examples, operating circuitry 116 is configured to ramp the pacing signals toward the high-rate pacing to assist first electrode 106 in the ventricular capture. Operating circuitry 116 control at least one of an amplitude, a frequency, or a chronological duration of a plurality of electrical pulses emitted by first electrode 106 to ramp the pacing signals. In some examples, operating circuitry 116 causes first electrode 106 to emit electric pulses configured to cause pacing of heart 102 at a first rate. Operating circuitry 116 may (e.g., subsequent to the first rate) cause first electrode 106 to emit electric pulses configured to cause pacing of heart 102 at a second rate greater than the first rate. Operating circuitry 116 may pause the conduction system pacing to evaluate if the conduction system pacing delivered by first electrode 106 has restored heart 102 to a normal sinus rhythm.

The technique includes causing, using operating circuitry 116, and following delivery of the pacing signals, and if the arrhythmia continues or another arrhythmia is detected by operating circuitry 116, defibrillation element 110 to deliver a shock to heart 102 (806). Defibrillation element 110 may transmit the shock to heart 102 to depolarize cardiac muscles and allow heart 102 (e.g., the sinoatrial node) to re-establish a proper rhythm. In examples, defibrillation element 110 causes a shock vector substantially across a ventricle of heart 102. The shock vector may extend, for example, from defibrillation element 110 to housing return electrode 121, return electrode 119, shock return electrode 176, and/or another electrode within medical system 100. In some examples, the shock vector extends from one electrode of defibrillation element 110 (e.g., one of defibrillation electrode 118 and/or sensing electrodes 120) to another electrode of defibrillation element 110 (e.g., another of defibrillation electrodes 118 and/or sensing electrodes 120).

In examples, device housing 122 supports a power supply 131 electrically connected to first electrode 106 and/or defibrillation element 110. Power supply 131 may provide power to first electrode 106 when first electrode 106 provides conduction system pacing to conduction system 117. Power supply 131 may provide power to defibrillation element 110 when defibrillation element 110 delivers a shock to heart 102. In examples, monitor device 101 receives power from an external power supply to deliver power to first electrode 106 and/or defibrillation element 110.

A first lead 108 (e.g., first lead distal portion 126) may support electrode support 107 and/or first electrode 106. A second lead 134 (e.g., second lead distal portion 136) may support defibrillation element 110. First lead 108 may define and/or include first link 103. Second lead 134 may define and/or include second link 105. In examples, the first lead 108 and/or second lead 134 passes through vasculature of patient 104 such that at least some portion (e.g., first lead distal portion 126 and/or second lead distal portion 136) resides within a chamber (e.g., a ventricle) of heart 102. First lead proximal portion 128 and/or second lead proximal portion 138 may be supported by monitor device 101 (e.g., device housing 122) when first lead distal portion 126 and/or second lead distal portion 136 passes through vasculature of patient 104. First lead 108 may support a first attachment structure 130 engaging heart tissue when the first lead distal portion 126 resides within the chamber. Second lead 134 may support second attachment structure 146 engaging heart tissue within a chamber when second lead distal portion 136 resides within the chamber. Medical system 100 may position defibrillation element 110 in proximity to an apex of a ventricle when first electrode 106 positions within or in the vicinity of target site 114, 115.

In some examples, unitary lead 152 (e.g., unitary lead distal portion 156) supports electrode support 107, first electrode 106 and defibrillation element 110. Unitary lead 152 may define and/or include first link 103 and/or second link 105. Unitary lead 152 may pass through vasculature of patient 104 such that at least some portion (e.g., unitary lead distal portion 156) resides within a chamber of the heart (e.g., a ventricle). Unitary lead proximal portion 158 may be supported by monitor device 101 (e.g., device housing 122) when unitary lead distal portion 156 passes through vasculature of patient 104. In examples, unitary lead 153 includes lead attachment structure 160. Lead attachment structure 160 may engage heart tissues of heart 102 to substantially establish and/or maintain first electrode 106 and/or electrode support 107 within the tissues of heart 102. In examples, unitary lead 152 positions defibrillation element 110 within a ventricle when lead attachment structure 160 engages heart tissues within the ventricle. In examples, unitary lead 152 positions defibrillation element 110 in proximity to an apex of the ventricle when lead attachment structure 160 engages tissues within the ventricle.

In examples, monitor device 101 (e.g., device housing 122) resides within a chamber of the heart (e.g., a ventricle). The device housing may support at least some portion of the operating circuitry. Monitor device 101 may position within the chamber when first electrode 106 positions in proximity to conduction system 117 (e.g., in proximity to target site 114, 115). Monitor device 101 (e.g., device housing 122) may support device lead 164 and/or defibrillation element 110 within the ventricle when first electrode 106 positions in proximity to conduction system 117. In examples, housing distal portion 165 supports electrode support 107 and/or first electrode 106. Device housing 122 (e.g., housing proximal portion 166) may support device lead distal portion 170. In examples, electrode support 107 defines and/or includes first link 103. Device lead 164 may define and/or include second link 105.

Device housing 122 (e.g., housing distal portion 165) may support device attachment structure 168. Device attachment structure 168 may engage heart tissue within a chamber of heart 102 when device housing 122 resides within the chamber. In examples, device lead 164 (e.g., device lead proximal portion 172) supports device lead attachment structure 174. Device lead attachment structure 174 may engage heart tissue within a chamber of heart 102 (e.g., a ventricle) when device lead 164 and/or device housing 122 resides within the chamber. In examples, device lead 164 positions defibrillation element 110 in the vicinity of an apex of a ventricle (e.g., an apex of the RV) when device lead attachment structure 174 engages tissues within the apex of the ventricle.

Various examples of the disclosure have been described. Any combination of the described systems, operations, or functions is contemplated. These and other examples are within the scope of the following claims.

The following examples are a non-limiting list of clauses in accordance with one or more techniques of this disclosure.

Example 1. A medical system, comprising: a first electrode configured to deliver pacing signals to a conduction system of a heart, wherein the first electrode is configured to capture the conduction system of the heart when the first electrode delivers the pacing signals and the first electrode is positioned within tissues of the heart; an electrode support supporting the first electrode, wherein the electrode support is configured to engage tissue of the heart to position the first electrode within the tissues of the heart; a defibrillation element configured to deliver a defibrillation shock to the heart of a patient, wherein the defibrillation element is configured to position within a ventricle of the heart; a monitor device including operating circuitry, wherein the operating circuitry is configured to: monitor one or physiological signals of the patient, detect a current or imminent arrhythmia using the one or more physiological signals, cause, in response to detecting the current or imminent arrhythmia, the first electrode to deliver the pacing signals to the conduction system of the heart, and cause, following delivery of the pacing signals, and if the arrhythmia continues or another arrhythmia is detected, the defibrillation element to deliver a shock to the heart of the patient.

Example 2. The medical system of example 1, wherein the pacing signals are configured to cause high rate pacing of the heart.

Example 3. The medical system of example 1 of example 2, wherein the operating circuitry is configured to determine an intrinsic heart rate of the heart, and wherein the pacing signals are configured to pace the heart at a heart rate greater than the intrinsic heart rate.

Example 4. The medical system of any of examples 1-3, wherein the operating circuitry is configured to: cause the first electrode to cease delivering the pacing signals, pause between causing the first electrode to cease and causing the defibrillation element to deliver the shock.

Example 5. The medical system of example 4, wherein the pause is at least partially defined by a chronological period.

Example 6. The medical system of example 4, wherein the pause is at least partially defined by the one or physiological signals.

Example 7. The medical system of any of examples 1-6, wherein the operating circuitry is configured to cause the first electrode to deliver the pacing signals over a time span, and wherein the pacing signals are configured to cause a heart rate of the heart to increase over the time span.

Example 8. The medical system of any of examples 1-7, wherein the operating circuitry is configured to cause the first electrode to emit a plurality of electrical pulses to cause the first electrode to deliver the pacing signals.

Example 9. The medical system of example 8, wherein the plurality of electrical pulses define one or more of an amplitude, a frequency, or a chronological duration, and wherein the operating circuitry is configured to control at least one of the amplitude, the frequency, or the chronological duration.

Example 10. The medical system of example 9, wherein the operating circuitry is configured to alter at least one of the amplitude, the frequency, or the chronological duration.

Example 11. The medical system of any of examples 1-10, wherein the pacing signals are configured to cause conduction system pacing of the heart at a first rate, and wherein the operating circuitry is configured to cause the first electrode to deliver second pacing signals to the heart, wherein the second pacing signals are configured to cause conduction system pacing of the heart at a second rate different than the first rate.

Example 12. The medical system of any of examples 1-11, wherein the electrode support is configured to position within the right ventricle, the left ventricle, or an atrium of the heart when the electrode support engages the tissues of the heart.

Example 13. The medical system of any of examples 1-12, wherein the monitor device includes a device housing supporting at least some portion of the operating circuitry.

Example 14. The medical system of any of examples 1-13, further comprising a return electrode, wherein the medical system is configured to at least one of: electrically couple the first electrode and the return electrode using at least one of blood of the patient or anatomical structures of the heart, or electrically couple the defibrillation element and the return electrode using at least one of the blood of the patient or the anatomical structures of the heart.

Example 15. The medical system of example 14, wherein the operating circuitry is configured to at least one of: electrically couple the first electrode and the return electrode, or electrically couple the defibrillation element and the return electrode.

Example 16. The medical system of example 14 or example 15, wherein the medical system is configured to position the return electrode relative to the defibrillation element to cause a shock vector from the defibrillation element to the return electrode to pass through a ventricular wall of the heart.

Example 17. The medical system of any of examples 1-16, wherein the operating circuitry is configured to cause a first electrode to deliver the pacing signals to at least one of a Bundle of His of the heart, a Left Bundle Branch of the heart, or Purkinje fibers of the heart.

Example 18. The medical system of any of examples 1-17, further comprising: a first lead supporting the first electrode, wherein the first lead includes a first conductor electrically connecting the first electrode and the operating circuitry. a second lead supporting the defibrillation element, wherein the second lead includes a second conductor electrically connecting the defibrillation element and the operating circuitry.

Example 19. The medical system of example 18, wherein the first lead includes a distal portion supporting the electrode support and a proximal portion supported by the device housing, and wherein the second lead includes a distal lead portion supporting the defibrillation element and a proximal lead portion supported by the device housing.

Example 20. The medical system of example 18 or example 19, wherein the distal portion is configured to position within the right ventricle, the left ventricle, or the atrium when the first electrode positions within the tissues of the heart, wherein the proximal portion is configured to position outside of the heart when the first electrode positions within the tissues of the heart, and wherein the device housing is configured to position outside the heart when the first electrode positions within the tissues of the heart.

Example 21. The medical system of any of examples 18-20, wherein the distal lead portion is configured to position within the ventricle of the heart when the defibrillation element positions within the ventricle of the heart, wherein the proximal lead portion is configured to position outside of the heart when the distal lead portion positions within the ventricle of the heart, and wherein the device housing is configured to position outside the heart when the distal lead portion positions within the ventricle of the heart.

Example 22. The medical system of any of examples 18-21, wherein the second lead is configured to allow a distal end of the second lead to position within an apex of the ventricle of the heart when the electrode support engages tissues of the heart.

Example 23. The medical system of any of examples 18-22, wherein the first lead supports a first attachment structure configured to engage heart tissues of the heart to substantially maintain the position of the first electrode within the tissues of the heart.

Example 24. The medical system of any of examples 18-23, wherein the second lead supports a second attachment structure configured to engage heart tissues of the heart to substantially maintain the position of the defibrillation element within the ventricle of the heart.

Example 25. The medical system of any of examples 1-17, further comprising a lead including a distal portion supporting the first electrode, the electrode support, and the defibrillation element, and including a proximal portion supported by device housing, wherein the distal portion is configured to allow the electrode support to position within the ventricle of the heart when the first electrode positions within the tissues of the heart, and wherein the distal portion is configured to position within an apex of the ventricle of the heart when the electrode support positions within the ventricle of the heart.

Example 26. The medical system of example 25, wherein the lead includes a first conductor electrically connecting the first electrode and the operating circuitry and includes a second conductor electrically connecting the defibrillation element and the operating circuitry.

Example 27. The medical system of example 25 or example 26, wherein the lead is a pre-shaped lead.

Example 28. The medical system of any of examples 25-27, wherein the proximal portion is configured to position outside of the heart when the distal portion positions within the ventricle of the heart, and wherein the device housing is configured to position outside the heart when the proximal portion positions outside the heart.

Example 29. The medical system of any of examples 25-28, wherein the lead supports an attachment structure configured to engage heart tissues of the heart to substantially maintain the position of the first electrode within the tissues of the heart.

Example 30. The medical system of any of examples 1-17, further comprising a lead supporting the defibrillation element, wherein the device housing supports the electrode support at a proximal end of the device housing, wherein the device housing is configured to position within the ventricle of the heart when the electrode support is positioned within the ventricle of the heart, and wherein the lead includes a distal portion supporting the defibrillation element and a proximal portion supported by the device housing.

Example 31. The medical system of example 30, wherein the lead is configured to allow a distal end of the lead to position within an apex of the ventricle of the heart when the first electrode positions within the ventricle of the heart.

Example 32. The medical system of example 30 or example 31, wherein the device housing supports a device attachment structure configured to engage heart tissues of the heart to substantially maintain the position of the first electrode within the tissues of the heart.

Example 33. The medical system of any of examples 30-32, wherein the lead supports a lead attachment structure configured to engage heart tissues of the heart to substantially maintain the position of the defibrillation element within the ventricle of the heart.

Example 34. A method of using a medical system, comprising: monitoring, using operating circuitry, one or physiological signals of a patient to detect a current or imminent arrhythmia using the one or more physiological signals; causing, using the operating circuitry, and in response to detecting the current or imminent arrhythmia, a first electrode to deliver pacing signals to a conduction system of a heart, wherein the first electrode is configured to capture the conduction system of the heart when the first electrode delivers the pacing signals and the first electrode is positioned within tissues of the heart, and wherein an electrode support supporting the first electrode is configured to engage tissue of the heart to position the first electrode within the tissues of the heart; and causing, using the operating circuitry, and following delivery of the pacing signals, and if the arrhythmia continues or another arrhythmia is detected by the operating circuitry, a defibrillation element to deliver a shock to the heart of the patient, wherein the defibrillation element is configured to position within a ventricle of the heart.

Example 35. The method of example 34, wherein the pacing signals are configured to cause high rate conduction system pacing of the heart.

Example 36. The method of example 34 or example 35, further comprising: determining, using the operating circuitry, an intrinsic heart rate of the heart, and causing, using the operating circuitry, the first electrode to deliver pacing signals configured to pace the heart at a heart rate greater than the intrinsic heart rate.

Example 37. The method of any of examples 34-36, further comprising: causing, using the operating circuitry, the first electrode to cease delivering the pacing signals; and pausing, by the operating circuitry, between causing the first electrode to cease causing the defibrillation element to deliver the shock.

Example 38. The method of example 37, wherein the pause is at least partially defined by a chronological period.

Example 39. The method of example 36, wherein the pause is at least partially defined by the one or physiological signals.

Example 40. The method of any of examples 34-39, wherein the operating circuitry causes the first electrode to deliver the pacing signals over a time span, wherein the pacing signals are configured to cause the heart to define an increasing heart rate over the time span.

Example 41. The method of any of examples 34-40, wherein the operating circuitry causes the first electrode to emit a plurality of electrical pulses to cause the first electrode to deliver the pacing signals.

Example 42. The method of example 41, wherein the operating circuitry controls at least one of an amplitude, a frequency, or a chronological duration defined by the plurality of electrical pulses.

Example 43. The method of example 42, wherein the operating circuitry is configured to alter at least one of the frequency, the amplitude, or the chronological duration defined by the plurality of electrical pulses.

Example 44. The method of any of examples 34-43, further comprising: causing, using the operating circuitry, the first electrode to deliver pacing signals configured to cause conduction system pacing of the heart at a first rate; and causing, using the operating circuitry, the first electrode to deliver pacing signals configured to cause conduction system pacing of the heart at a second rate different than the first rate.

Example 45. The method of any of examples 34-44, further comprising engaging, using the electrode support, tissues of the heart when the electrode support is positioned within a right ventricle, the left ventricle, or an atrium of the heart.

Example 46. The method of any of examples 34-45, further comprising supporting, using a device housing of the monitor device, at least some portion of the operating circuitry.

Example 47. The method of any of examples 34-46, further comprising at least one of: electrically connecting, by the medical system, the first electrode and a return electrode using at least one of blood of the patient or anatomical structures of the heart, wherein the return electrode is electrically connected to the operating circuitry; or electrically connecting, by the medical system, the defibrillation element and the return electrode using at least one of the blood of the patient or the anatomical structures of the heart.

Example 48. The method of any of examples 34-47, further comprising: supporting, using a first lead having a distal portion and a proximal portion, the first electrode using the distal portion, wherein the first lead includes a first conductor electrically connecting the first electrode and the operating circuitry, and wherein the proximal portion supported by the device housing; and supporting, using a second lead having a distal lead portion and a proximal lead portion, the defibrillation element using the distal lead portion, wherein the second lead includes a second conductor electrically connecting the defibrillation element and the operating circuitry, and wherein the proximal lead portion is supported by the device housing.

Example 49. The method of example 48, wherein the distal portion is positioned within a right ventricle, a left ventricle, or an atrium of the heart and the proximal portion is positioned outside of the heart when the first electrode positions within the tissues of the heart, wherein the distal lead portion is positioned within the ventricle of the heart and the proximal lead portion is positioned outside of the heart when the when the defibrillation element positions within the ventricle of the heart, and wherein the device housing is positioned outside the heart when the first electrode positions within the tissues of the heart and the defibrillation element positions within the ventricle of the heart.

Example 50. The method of example 48 or example 49, further comprising: engaging, using a first attachment structure supported by the first lead, heart tissues of the heart at a first location to substantially maintain the position of the first electrode within the tissues of the heart; and engaging, using a second attachment structure supported by the second lead, heart tissues of the heart at a second location to substantially maintain the position of the defibrillation element within the ventricle of the heart.

Example 51. The method of example 50, wherein the first location is within the ventricle of the heart, and wherein the second location is within an apex of the ventricle of the heart.

Example 52. The method of any of examples 34-47, further comprising: supporting, using a lead including a distal portion and a proximal portion, the first electrode, the electrode support, and the defibrillation element using the distal portion, wherein the proximal portion is supported by the device housing; positioning, using the distal portion, the electrode support within the ventricle of the heart to when the first electrode positions within the tissues of the heart; and positioning, using the lead, the distal portion within an apex of the ventricle of the heart when the electrode support positions within the ventricle of the heart.

Example 53. The method of example 52, further comprising positioning the proximal portion and the device housing outside of the heart when the distal portion positions within the ventricle of the heart.

Example 54. The method of example 52 or claim 53, further comprising engaging, using a first attachment structure, heart tissues of the heart at a first location to substantially maintain the position of the first electrode within the tissues of the heart.

Example 55. The method of any of examples 52-54, further comprising engaging, using a second attachment structure, heart tissues of the heart at a second location to substantially maintain the position of the defibrillation element within the ventricle of the heart.

Example 56. The medical system of any of examples 34-47, further comprising supporting, using the device housing of claim 45, a lead supporting the defibrillation element, wherein the wherein the device housing supports the electrode support at a proximal end of the device housing, wherein the device housing is configured to position within the ventricle of the heart when the electrode support is positioned within the ventricle of the heart, and wherein the lead includes a distal portion supporting the defibrillation element and a proximal portion supported by the device housing.

Example 57. The method of example 56, further comprising positioning, using the lead, a distal end of the lead within an apex of the ventricle of the heart.

Example 58. The method of example 56 or example 57, further comprising: engaging, using at least one of the electrode support or a device attachment structure supported by the device housing, heart tissues of the heart at a first location within the ventricle of the heart to substantially maintain the position of the first electrode within the tissues of the heart; and engaging, using a lead attachment structure supported by the lead, heart tissues of the heart at a second location within the ventricle of the heart to substantially maintain the position of the defibrillation element within the ventricle.

Example 59. The method of any of examples 33-58, wherein causing the first electrode to deliver pacing signals to the conduction system of the heart includes causing the first electrode to deliver the pacing signals to at least one of a Bundle of His of the heart, a Left Bundle Branch of the heart, or Purkinje fibers of the heart.

1: A medical system, comprising:
a first electrode configured to deliver pacing signals to a conduction system of a heart, wherein the first electrode is configured to capture the conduction system of the heart when the first electrode delivers the pacing signals and the first electrode is positioned within tissues of the heart;
an electrode support supporting the first electrode, wherein the electrode support is configured to engage tissue of the heart to position the first electrode within the tissues of the heart;
a defibrillation element configured to deliver a defibrillation shock to the heart of a patient, wherein the defibrillation element is configured to position within a ventricle of the heart;
a monitor device including operating circuitry, wherein the operating circuitry is configured to:
monitor one or physiological signals of the patient,
detect a current or imminent arrhythmia using the one or more physiological signals, cause, in response to detecting the current or imminent arrhythmia, the first electrode to deliver the pacing signals to the conduction system of the heart, and
cause, following delivery of the pacing signals, and if the arrhythmia continues or another arrhythmia is detected, the defibrillation element to deliver a shock to the heart of the patient.

2: The medical system of claim 1, wherein the pacing signals are configured to cause high rate pacing of the heart.

3: The medical system of claim 1, wherein the operating circuitry is configured to:
cause the first electrode to cease delivering the pacing signals,
pause between causing the first electrode to cease and causing the defibrillation element to deliver the shock.

4: The medical system of claim 3, wherein the pause is at least partially defined by one or more of a chronological period or by the one or more physiological signals.

5: The medical system of claim 1, wherein the operating circuitry is configured to cause the first electrode to deliver the pacing signals over a time span, and wherein the pacing signals are configured to cause a heart rate of the heart to increase over the time span.

6: The medical system of claim 1, wherein the pacing signals are configured to cause conduction system pacing of the heart at a first rate, and wherein the operating circuitry is configured to cause the first electrode to deliver second pacing signals to the heart, wherein the second pacing signals are configured to cause conduction system pacing of the heart at a second rate different than the first rate.

7: The medical system of claim 1, wherein the operating circuitry is configured to cause a first electrode to deliver the pacing signals to at least one of a Bundle of His of the heart, a Left Bundle Branch of the heart, or Purkinje fibers of the heart.

8: The medical system of claim 1, further comprising:
a first lead supporting the first electrode, wherein the first lead includes a first conductor electrically connecting the first electrode and the operating circuitry;
a second lead supporting the defibrillation element, wherein the second lead includes a second conductor electrically connecting the defibrillation element and the operating circuitry.

9: The medical system of claim 8,
wherein the distal portion is configured to position within the right ventricle, the left ventricle, or the atrium when the first electrode positions within the tissues of the heart,
wherein the proximal portion is configured to position outside of the heart when the first electrode positions within the tissues of the heart, and
wherein the device housing is configured to position outside the heart when the first electrode positions within the tissues of the heart.

10: The medical system of claim 8,
wherein the distal lead portion is configured to position within the ventricle of the heart when the defibrillation element positions within the ventricle of the heart,
wherein the proximal lead portion is configured to position outside of the heart when the distal lead portion positions within the ventricle of the heart, and
wherein the device housing is configured to position outside the heart when the distal lead portion positions within the ventricle of the heart.

11: The medical system of claim 1, further comprising a lead including a distal portion supporting the first electrode, the electrode support, and the defibrillation element, and including a proximal portion supported by device housing,
wherein the distal portion is configured to allow the electrode support to position within the ventricle of the heart when the first electrode positions within the tissues of the heart, and
wherein the distal portion is configured to position within an apex of the ventricle of the heart when the electrode support positions within the ventricle of the heart.

12: The medical system of claim 11,
wherein the proximal portion is configured to position outside of the heart when the distal portion positions within the ventricle of the heart, and
wherein the device housing is configured to position outside the heart when the proximal portion positions outside the heart.

13: The medical system of claim 11, wherein the lead supports an attachment structure configured to engage heart tissues of the heart to substantially maintain the position of the first electrode within the tissues of the heart.

14: The medical system of claim 1, further comprising a lead supporting the defibrillation element,
wherein the device housing supports the electrode support at a proximal end of the device housing,
wherein the device housing is configured to position within the ventricle of the heart when the electrode support is positioned within the ventricle of the heart, and
wherein the lead includes a distal portion supporting the defibrillation element and a proximal portion supported by the device housing.

15: The medical system of claim 14, wherein the device housing supports a device attachment structure configured to engage heart tissues of the heart to substantially maintain the position of the first electrode within the tissues of the heart.

16: A medical system, comprising:
a first electrode configured to deliver pacing signals to a conduction system of a heart, wherein the first electrode is configured to capture the conduction system of the heart when the first electrode delivers the pacing signals and the first electrode is positioned within tissues of the heart;
an electrode support supporting the first electrode, wherein the electrode support is configured to engage tissue of the heart to position the first electrode within the tissues of the heart;
a defibrillation element configured to deliver a defibrillation shock to the heart of a patient, wherein the defibrillation element is configured to position within a ventricle of the heart;
a monitor device including operating circuitry, wherein the operating circuitry is configured to:
monitor one or physiological signals of the patient,
detect a current or imminent arrhythmia using the one or more physiological signals,
determine an intrinsic heart rate of the heart.
cause, in response to detecting the current or imminent arrhythmia, the first electrode to deliver the pacing signals to the conduction system of the heart, wherein the pacing signals are configured to pace the heart at a heart rate greater than the intrinsic heart rate to cause high rate pacing of the heart, and
cause, following delivery of the pacing signals, and if the arrhythmia continues or another arrhythmia is detected, the defibrillation element to deliver a shock to the heart of the patient.

17: The medical system of claim 17, wherein the operating circuitry is configured to cause the first electrode to deliver the pacing signals over a time span, and wherein the pacing signals are configured to cause a heart rate of the heart to increase over the time span.

18: The medical system of claim 17, wherein the operating circuitry is configured to:
cause the first electrode to cease delivering the pacing signals, and
pause between causing the first electrode to cease and causing the defibrillation element to deliver the shock.

19: A method of using a medical system, comprising:
monitoring, using operating circuitry, one or physiological signals of a patient to detect a current or imminent arrhythmia using the one or more physiological signals;
causing, using the operating circuitry, and in response to detecting the current or imminent arrhythmia, a first electrode to deliver pacing signals to a conduction system of a heart,
wherein the first electrode is configured to capture the conduction system of the heart when the first electrode delivers the pacing signals and the first electrode is positioned within tissues of the heart, and
wherein an electrode support supporting the first electrode is configured to engage tissue of the heart to position the first electrode within the tissues of the heart; and
causing, using the operating circuitry, and following delivery of the pacing signals, and if the arrhythmia continues or another arrhythmia is detected by the operating circuitry, a defibrillation element to deliver a shock to the heart of the patient, wherein the defibrillation element is configured to position within a ventricle of the heart.

20: The method of claim 19, wherein the pacing signals are configured to cause high rate conduction system pacing of the heart.
